(19) United States
(12) Patent Application Publication
(10) Pub. No.: US 2022/0396774 A1
(43) Pub. Date: Dec. 15, 2022

(54) COMPOSITIONS AND METHODS FOR GENERATING INSULIN-PRODUCING BETA CELLS

(71) Applicant: BETALIN THERAPEUTICS LTD., Nahariya (IL)

(72) Inventors: Orit GOLDMAN, Jerusalem (IL); Nikolai KUNICHER, Pardesia (IL); Manuela VECSLER, Petah Tikva (IL); Avi TREVES, Mevasseret Zion (IL)

(73) Assignee: BETALIN THERAPEUTICS LTD., Nahariya (IL)

(21) Appl. No.: 17/622,436

(22) PCT Filed: Jul. 1, 2020
(86) PCT No.: PCT/IL2020/050735

§ 371 Date: Dec. 23, 2021

Related U.S. Application Data
(60) Provisional application No. 62/869,038, filed on Jul. 1, 2019.

(51) Int. Cl. C12N 5/071; C12N 5/074; A61F 2/02

(52) U.S. Cl. CPC C12N 5/0677; C12N 5/0607; A61F 2/022; C12N 2506/22

(57) ABSTRACT

Compositions and methods for generating insulin-producing beta cells from pluripotent stem cells are provided. The compositions and methods of the present invention involve stepwise differentiation while the differentiating cells are cultured on a lung tissue-derived acellular scaffold.

### FIELD OF THE INVENTION

The present invention relates to compositions and methods for generating insulin-producing beta cells from pluripotent stem cells. The compositions and methods of the present invention involve stepwise differentiation and optionally proliferation while the cells are cultured on a lung tissue-derived acellular scaffold.

### BACKGROUND OF THE INVENTION

Induced pluripotent stem cells (iPSC) as well as embryonic stem cells (ESC) possess unique properties of self-renewal and capability to differentiate to many types of cells. It has been proposed to use such cells for generating insulin-producing beta cells for transplantation to diabetes mellitus (type 1 diabetes) patients.

A variety of in vitro differentiation protocols have been developed based on an initial work of Rezania et al., 2014, Nat Biotechnol, 32(11):1121-33, which can successfully differentiate human ESC/iPSCs into monohormonal insulin-producing cells that phenotypically and functionally resemble mature beta cells (for example Pagliuca et al., 2014, Cell., 159:428-39; and Russ et al., 2015, EMBO J., 34:1759-72). An important feature of these protocols is the generation of pancreatic progenitors that co-express PDX1 and NKX6.1, through which improved yields of insulin-producing cells can be obtained. Although the generated insulin-producing cells reverse diabetes after transplantation in rodent models and demonstrate glucose-responsiveness on glucose-stimulated insulin secretion (GSIS) assays in vitro, perfusion assays demonstrated that the insulin secretion kinetics and mitochondrial respiration of these cells are functionally immature. This immaturity raises questions as to the safety of therapeutic use of the cells, due to tumorigenic risks of any undifferentiated cells remaining following the differentiation process and due to possible auto/alloimmune reaction against the cells following transplantation.

In their native environment, beta cells are located within aggregates known as the islets of Langerhans, which are comprised of endocrine cells and extracellular matrix (ECM) molecules, and are found embedded in pancreatic tissue. Within this three-dimensional environment, beta cells experience cell-matrix and cell-cell interactions. Previous studies have shown evidence of the critical role played by the extracellular matrix (ECM) in pancreatic cell proliferation and development. Cell-matrix interactions have been shown to improve beta cell proliferation, insulin secretion and islet development (Wang et al., 2017, Stem Cells Dev., 26(6):394-404; Weber et al., 2008, Tissue Eng Part A, 14:1959-1968; Hammar et al., 2004, Diabetes, 53: 2034-2041).

A 3D cell culture is an artificially created environment in which cells are permitted to grow or interact with their surroundings in all three dimensions. The cells are often embedded within a material where they can migrate and experience cell-matrix interactions and cell-cell contacts in all three dimensions. 3D cell culture platforms represent an improved approach to in vitro cell culture and differentiation that better captures the native tissue environment. Several studies have used various artificial scaffolds, such as PES, PLLA/PVA and PCL/PVA scaffolds, to obtain human insulin-producing cells from iPSCs (Enderami et al., 2018, Artif Cells Nanomed Biotechnol., 2:1-8; Abazari et al., 2018, Gene, 5:50-57; Mansour et al., Artif Cells Nanomed Biotechnol., 2:1-7). However, such scaffolds have limitations, such as non-biodegradability, low potential to attract cells that penetrate into the scaffold's structure, and/or hydrophobicity, resulting in poor cell attachment. Such scaffolds lack the structure and properties of natural tissue microenvironment.

Sionov et al., 2015, Tissue Eng Part A, 21(21-22): 2691-702 report the preparation of endocrine micro-pancreata (EMPs) that are made up of acellular pancreas-derived or lung-derived micro-scaffolds seeded with human intact or enzymatically dissociated pancreatic islets.

U.S. Pat. No. 7,297,540 discloses the use of micro-organs (MOs), which are explants of tissue that retain the basic cell-cell, cell-matrix and cell-stroma architecture of the originating tissue, as a (continuous) source of adult stem cells; the use of the natural multi-signaling micro-environment of micro-organs to induce differentiation of stem cells; and the use of the natural three-dimensional structure of an MO acellular matrix as a scaffold for seeding stem cells of adult or embryonic origin.

U.S. Pat. No. 10,093,896 discloses a composition of matter comprising a devitalized, acellular tissue-derived scaffold seeded with differentiated cells, particularly pancreatic islets or pancreatic islet cells, wherein the cells can maintain cell-specific function or structure in culture on the scaffold. Methods of generating same and uses thereof are also disclosed.

There is a need to improve the quality, functionality and maturity of beta cells generated in vitro from pluripotent stem cells.

### SUMMARY OF THE INVENTION

The present invention provides compositions, methods and kits for producing high quality human beta cells differentiated from human pluripotent stem cells. The compositions, methods and kits of the present invention employ stepwise differentiation in which a plurality of differentiation factors are sequentially applied, while the differentiating cells are cultured on a lung tissue-derived acellular scaffold, also referred to as acellular micro-organ matrix (MOM). All differentiation steps may or may not be accompanied by cell proliferation.

The inventors of the present invention utilized a stepwise differentiation process that is typically carried out in a 2D cell culture, in which a plurality of differentiation factors are sequentially applied. The inventors have found that by carrying out the process while the differentiating cells are cultured on a lung-tissue derived three-dimensional scaffold (3D), beta cells with improved functionality and maturity can be obtained.

The present invention discloses for the first time the generation of beta cells by cell differentiation on a non-homologous tissue scaffold, namely, a tissue scaffold derived from a tissue other than the pancreas, and particularly on a lung tissue-derived scaffold. Previous reports showed that a scaffold derived from a natural tissue affects the differentiation of cells cultured thereon and directs it towards the tissue from which the scaffold is derived. Surprisingly, the inventors of the present invention found that beta cells can be obtained by differentiation on a lung tissue-derived scaffold, which are characterized by improved insulin production compared to cells produced using the same differentiation procedure in a 2-dimensional cell culture. Thus, not only that the lung source of the scaffold did not negatively affect the differentiation process, but rather it resulted in beta cells with improved properties.

Advantageously, the lung tissue-derived 3D scaffold provides an enormous surface area lined by basement membrane, which resembles the natural microenvironment of beta cells in the pancreas. A lung tissue-derived scaffold was found to be advantageous even over a pancreas-derived scaffold. A pancreas-derived scaffold mainly contains matrix derived from the exocrine portion of the pancreas rather than the endocrine part, which contains the islets of Langerhans, as the endocrine part constitutes only about 1-2% of the pancreas. The endocrine portion of the pancreas is characterized by a highly dense vasculature surrounded by basement membrane. Thus, a lung tissue-derived scaffold better mimics the islet tissue natural microenvironment.

The beta cells resulting from differentiation on lung tissue-derived scaffolds as disclosed herein are fully mature (for example, they express MAFA transcription factor that is expressed in adult beta cells and is absent in developing beta cells and other pancreatic cells), and secrete insulin in response to glucose stimulation at higher levels compared to cells produced by differentiation in a 2D cell culture. Importantly, the beta cells obtained as disclosed herein secrete the insulin in a regulated bi-phasic manner in response to glucose stimulation, as evidenced by dynamic glucose-responsive insulin secretion assays examining insulin secretion in response to glucose over time. This is in contrast to cells produced by differentiation in a 2D cell culture, as exemplified hereinbelow. The regulated insulin secretion in response to glucose indicates that the obtained cells are functionally mature. In addition, differentiation on lung tissue-derived scaffolds as disclosed herein resulted in more cells expressing insulin compared to differentiation in a 2D culture. Thus, the methods of differentiation disclosed herein provide higher yield of fully differentiated beta cells, as well as increased and improved insulin secretion.

According to some embodiments, the lung tissue-derived scaffold is from a non-human source (e.g., porcine) and the differentiating cells are human. Surprisingly, as exemplified herein below, a non-human lung tissue-derived scaffold did not negatively affect the differentiation of human cells of the pancreatic lineage into beta cells.

According to one aspect, the present invention provides a method for generating a population of insulin-producing beta cells, the method comprising:

(a) seeding progenitor cells of the pancreatic lineage on a devitalized, acellular, lung tissue-derived three-dimensional scaffold; and

(b) differentiating the progenitor cells of the pancreatic lineage to beta cells by a stepwise differentiation comprising sequentially applying a plurality of differentiation factors, wherein the stepwise differentiation is carried out on the lung tissue-derived three-dimensional scaffold such that the cells remain on said scaffold throughout the differentiation process, thereby generating a population of insulin-producing beta cells.

In some embodiments, the method further comprises differentiating pluripotent stem cells to the progenitor cells of the pancreatic lineage in a 2D cell culture prior to step (a).

In some embodiments, the progenitor cells of the pancreatic lineage are selected from the group consisting of definitive endoderm cells, primitive gut cells, posterior foregut cells, pancreatic endoderm cells, pancreatic progenitor 1 (PP1) cells, endocrine pancreatic progenitor (PP2) cells and endocrine precursor cells (also called pancreatic endocrine progenitor cells). Each possibility represents a separate embodiment of the present invention.

In some embodiments, the progenitor cells of the pancreatic lineage are selected from the group consisting of definitive endoderm cells, primitive gut cells, posterior foregut cells, pancreatic endoderm cells and endocrine precursor cells. Each possibility represents a separate embodiment of the present invention.

In additional embodiments, the progenitor cells of the pancreatic lineage are selected from the group consisting of definitive endoderm cells, primitive gut cells, posterior foregut cells, pancreatic progenitor 1 (PP1) cells, endocrine pancreatic progenitor (PP2) cells and endocrine precursor cells (also called pancreatic endocrine progenitor cells). Each possibility represents a separate embodiment of the present invention.

In some particular embodiments, the progenitor cells of the pancreatic lineage are pancreatic endoderm cells. In some embodiments, the progenitor cells of the pancreatic lineage are pancreatic endoderm cells, and the sequentially applying a plurality of differentiation factors comprises:

(i) culturing the scaffold seeded with the pancreatic endoderm cells in a medium comprising one or more endocrine precursor differentiation factor, to obtain endocrine precursor cells on the scaffold; and

(ii) culturing the scaffold with the endocrine precursor cells in a medium comprising one or more beta cell differentiation factor, to obtain beta cells on the scaffold.

In some embodiments, the progenitor cells of the pancreatic lineage are pancreatic endoderm cells, and the method further comprises differentiating pluripotent stem cells to pancreatic endoderm cells in a 2D cell culture prior to step (a).

In some embodiments, the method further comprises seeding at least one type of supporting cells selected from endothelial cells and mesenchymal stem cells (MSCs) on the scaffold, and carrying out the differentiation process while the supporting cells are co-cultured with the differentiating cells on the scaffold.

In some particular embodiments, the method further comprises seeding both endothelial cells and mesenchymal stem cells (MSCs) on the scaffold, and carrying out the differentiation process while the endothelial cells and MSCs are co-cultured with the differentiating cells on the scaffold.

According to another aspect, the present invention provides a composition for generating insulin-producing beta cells, comprising:

(i) a devitalized, acellular, lung tissue-derived three-dimensional scaffold; and

(ii) progenitor cells of the pancreatic lineage seeded on said scaffold.

In some embodiments, the progenitor cells of the pancreatic lineage are selected from the group consisting of definitive endoderm cells, primitive gut cells, posterior foregut cells, pancreatic endoderm cells, pancreatic progenitor 1 (PP1) cells, endocrine pancreatic progenitor (PP2) cells and endocrine precursor cells (also called pancreatic endocrine progenitor cells). Each possibility represents a separate embodiment of the present invention.

In some embodiments, the progenitor cells of the pancreatic lineage are selected from the group consisting of definitive endoderm cells, primitive gut cells, posterior foregut cells, pancreatic endoderm cells and endocrine precursor cells. Each possibility represents a separate embodiment of the present invention.

In additional embodiments, the progenitor cells of the pancreatic lineage are selected from the group consisting of definitive endoderm cells, primitive gut cells, posterior foregut cells, pancreatic progenitor 1 (PP1) cells, endocrine pancreatic progenitor (PP2) cells and endocrine precursor cells (also called pancreatic endocrine progenitor cells). Each possibility represents a separate embodiment of the present invention.

In some particular embodiments, the progenitor cells of the pancreatic lineage are pancreatic endoderm cells.

In some embodiments, the composition further comprises at least one type of supporting cells selected from endothelial cells and mesenchymal stem cells (MSCs) seeded on the scaffold.

In some particular embodiments, the composition further comprises both endothelial cells and MSCs seeded on the scaffold.

According to a further aspect, the present invention provides a method for generating insulin-producing beta cells, comprising:

(a) providing a devitalized, acellular, lung tissue-derived three-dimensional scaffold seeded with progenitor cells of the pancreatic lineage according to the present invention; and

b) differentiating the progenitor cells of the pancreatic lineage to beta cells by a stepwise differentiation, wherein the stepwise differentiation is carried out on the lung tissue-derived three-dimensional scaffold such that the differentiating cells remain on said scaffold throughout the differentiation process.

In some embodiments, the scaffold in step (a) is further seeded with at least one type of supporting cells selected from endothelial cells and mesenchymal stem cells (MSCs), and wherein the stepwise differentiation is carried out on the scaffold in the presence of the supporting cells.

In some particular embodiments, the scaffold in step (a) is further seeded with both endothelial cells and mesenchymal stem cells (MSCs), and wherein the stepwise differentiation is carried out on the scaffold in the presence of the endothelia cells and the MSCs.

According to a further aspect, the present invention provides a kit for generating insulin-producing beta cells, comprising:

(i) a devitalized, acellular, lung tissue-derived three-dimensional scaffold;

(ii) a plurality of differentiation factors for conducting a stepwise differentiation of progenitor cells of the pancreatic lineage to beta cells; and

iii) an instruction manual specifying instructions for carrying out a stepwise differentiation on the scaffold such that cells remain on said scaffold throughout the differentiation process.

In some embodiments, the scaffold is pre-seeded with progenitor cells of the pancreatic lineage selected from the group consisting of: definitive endoderm cells, primitive gut cells, posterior foregut cells, pancreatic endoderm cells, pancreatic progenitor 1 (PP1) cells, endocrine pancreatic progenitor (PP2) cells and endocrine precursor cells (also called pancreatic endocrine progenitor cells). Each possibility represents a separate embodiment of the present invention.

In some embodiments, the scaffold is pre-seeded with progenitor cells of the pancreatic lineage selected from the group consisting of: definitive endoderm cells, primitive gut cells, posterior foregut cells, pancreatic endoderm cells and endocrine precursor cells. Each possibility represents a separate embodiment of the present invention.

In some embodiments, the scaffold is pre-seeded with progenitor cells of the pancreatic lineage selected from the group consisting of: definitive endoderm cells, primitive gut cells, posterior foregut cells, pancreatic progenitor 1 (PP1) cells, endocrine pancreatic progenitor (PP2) cells and endocrine precursor cells (also called pancreatic endocrine progenitor cells). Each possibility represents a separate embodiment of the present invention.

In some embodiments, the instruction manual further specifies instructions for seeding the progenitor cells of the pancreatic lineage on the scaffold prior to the stepwise differentiation. In some embodiments, the progenitor cells of the pancreatic lineage are selected from the group consisting of definitive endoderm cells, primitive gut cells, posterior foregut cells, pancreatic endoderm cells, pancreatic progenitor 1 (PP1) cells, endocrine pancreatic progenitor (PP2) cells and endocrine precursor cells. Each possibility represents a separate embodiment of the present invention.

In some embodiments, the instructions manual further specifies instructions for seeding progenitor cells of the pancreatic lineage selected from the group consisting of: definitive endoderm cells, primitive gut cells, posterior foregut cells, pancreatic endoderm cells and endocrine precursor cells. Each possibility represents a separate embodiment of the present invention.

In other embodiments, the instruction manual further specifies instructions for seeding progenitor cells of the pancreatic lineage selected from the group consisting of: definitive endoderm cells, primitive gut cells, posterior foregut cells, pancreatic progenitor 1 (PP1) cells, endocrine pancreatic progenitor (PP2) cells and endocrine precursor cells (also called pancreatic endocrine progenitor cells) on the scaffold prior to the stepwise differentiation. Each possibility represents a separate embodiment of the present invention.

In some embodiments, the kit further comprises a plurality of differentiation factors for conducting a stepwise differentiation of pluripotent stem cells into the progenitor cells of the pancreatic lineage in a 2D cell culture prior to seeding on the scaffold. In some embodiments, the instruction manual further specifies instructions for carrying out a stepwise differentiation of pluripotent stem cells into the progenitor cells of the pancreatic lineage in a 2D cell culture prior to seeding on the scaffold.

In some embodiments, the kit further comprises at least one type of supporting cells selected from endothelial cells and mesenchymal stem cells (MSCs) seeded on the scaffold.

In some particular embodiments, the kit further comprises both endothelial cells and MSCs seeded on the scaffold.

In some embodiments, the kit further comprises one or more cell culture medium.

According to a further aspect, the present invention provides a method for producing an artificial micro-organ, the method comprising:

(a) seeding progenitor cells of the pancreatic lineage on a devitalized, acellular, lung tissue-derived three-dimensional scaffold; and

(b) differentiating the progenitor cells of the pancreatic lineage to insulin-producing beta cells by a stepwise differentiation in which a plurality of differentiation factors are sequentially applied, wherein the stepwise differentiation is carried out on the lung tissue-derived three-dimensional scaffold such that the cells remain on said scaffold throughout the differentiation process,

thereby obtaining an artificial micro-organ comprising the insulin-producing beta cells cultured on the lung tissue-derived three-dimensional scaffold and maintaining glucose-responsive insulin secretion when cultured on said scaffold.

In some embodiments, the progenitor cells of the pancreatic lineage are selected from the group consisting of definitive endoderm cells, primitive gut cells, posterior foregut cells, pancreatic endoderm cells, pancreatic progenitor 1 (PP1) cells, endocrine pancreatic progenitor (PP2) cells and endocrine precursor cells (also called pancreatic endocrine progenitor cells). Each possibility represents a separate embodiment of the present invention.

In some embodiments, the progenitor cells of the pancreatic lineage are selected from the group consisting of definitive endoderm cells, primitive gut cells, posterior foregut cells, pancreatic endoderm cells and endocrine precursor cells. Each possibility represents a separate embodiment of the present invention.

In additional embodiments, the progenitor cells of the pancreatic lineage are selected from the group consisting of definitive endoderm cells, primitive gut cells, posterior foregut cells, pancreatic progenitor 1 (PP1) cells, endocrine pancreatic progenitor (PP2) cells and endocrine precursor cells (also called pancreatic endocrine progenitor cells). Each possibility represents a separate embodiment of the present invention.

In some embodiments, the method further comprises seeding at least one type of supporting cells selected from endothelial cells and mesenchymal stem cells (MSCs) on the scaffold, and carrying out the differentiation process while the supporting cells are co-cultured with the differentiating cells on the scaffold.

In some particular embodiments, the method further comprises seeding both endothelial cells and mesenchymal stem cells (MSCs) on the scaffold, and carrying out the differentiation process while the endothelial cells and MSCs are co-cultured with the differentiating cells on the scaffold.

According to a further aspect, the present invention provides an artificial micro-organ comprising a lung tissue-derived three-dimensional scaffold and insulin-producing beta cells cultured thereon, produced by the method of the present invention.

According to a further aspect, the present invention provides a method for treating diabetes in a subject in a need thereof, the method comprising transplanting in the subject a therapeutically effective amount of an artificial micro-organ produced by the method of the present invention.

In some embodiments, the diabetes is type I diabetes. In other embodiments, the diabetes is type II diabetes. In other embodiments the diabetes is caused by pancreatitis or other types of damage to the pancreas.

In some embodiments, the source of progenitor cells of the pancreatic lineage is autologous to the treated subject.

Other objects, features and advantages of the present invention will become clear from the following description, examples and drawings.

### BRIEF DESCRIPTION OF THE FIGURES

FIG. 1. Differentiation procedures. (A) Standard differentiation protocols in a 2D culture; (B) Differentiation procedure according to Example 1 hereinbelow.

FIG. 2. Differentiation procedure according to Example 2 hereinbelow from pluripotent stem cells (iPSC) to insulin-producing beta cells (IB), through: definitive endoderm, primitive gut, posterior foregut, pancreatic endoderm and endocrine precursor cell (endo. cell).

FIG. 3. FACS analysis of expression of the definitive endoderm markers CXCR4 and c-kit (Day 4 of the differentiation protocol according to Example 2 hereinbelow).

FIG. 4. Insulin expression by beta cells differentiated according to Example 2 hereinbelow. (A) qPCR analysis of INS mRNA expression in cells that were differentiated in a 2D culture; (B) Insulin/DAPI staining of cells that were differentiated in a 2D culture, 20-fold magnification; (C) Insulin/DAPI staining of cells that completed the differentiation on MOMs, 20-fold magnification.

FIG. 5. Glucose stimulated insulin secretion (GSIS) assay by beta cells differentiated according to Example 2 hereinbelow. (A) Cells that were seeded on MOMs on Day 15 and completed the differentiation on MOMs; (B) Cells that were grown in a 2D culture and clustered between Day 21-Day 24; (C) Cells grown as monolayers in a 2D culture plate.

### DETAILED DESCRIPTION OF THE INVENTION

The present invention is directed to the differentiation of pluripotent stem cells to functional insulin-producing beta cells using a natural matrix from decellularized lung tissue. The lung tissue-derived matrix preserves its complex tissue structure, which is similar in its complexity to the structural micro-environment of beta cells in the pancreas, with a large surface area lined by basal membrane.

As disclosed herein, the lung tissue-derived scaffold is seeded with progenitor cells of the pancreatic lineage and the progenitor cells are induced to differentiate to beta cells by a stepwise differentiation in which a plurality of differentiation factors are sequentially applied. The inventors of the present invention have shown that by carrying out the differentiation process on the scaffold according to the present invention, improved beta cells can be obtained compared to beta cells obtained by differentiation in a 2D culture.

Surprisingly, the lung tissue source of the scaffold did not negatively affect the differentiation to beta cells, and even resulted in improved beta cell differentiation.

Scaffold

As used herein, the term “scaffold” refers to a 3-dimensional matrix upon which cells may be cultured. Scaffolds according to the present invention are prepared from explants of tissue of microscopic thickness, also termed “micro-organs” (MOs). Micro-organs retain the basic cell-cell, cell-matrix and cell-stroma architecture of the originating tissue. To obtain a devitalized, acellular tissue-derived scaffold according to the present invention, the micro-organ explants are treated to remove cells, resulting in “micro-organ derived matrices”, abbreviated “MOMs”.

As used herein, the terms “devitalized” and “acellular” refer to a tissue or structure treated to remove living, cellular (including genetic material) mass. Devitalized, acellular micro-organs are micro-organ explants which essentially no longer comprise any cells or other living matter, do not reproduce, do not require a supply of nutrients or gas, and are essentially inert. In some embodiments, cells are killed and then removed from the tissue, but cells can be removed without prior killing. Dead cells may be allowed to slough off in liquid, or may be chemically or mechanically removed.

Methods for devitalization of tissue suitable for use with the present invention include thermal devitalization, irradiation, chemical stripping of cells by alkaline or acid treatment, hyperosmotic or hypo-osmotic devitalization, mechanical devitalization, detergents, organic solvents, combinations thereof and the like. It will be appreciated that inasmuch as the goal of devitalization is to provide an acellular scaffold for culture of cells, methods of devitalization suitable for use with the present invention are selected so as not to disrupt the structural and biochemical integrity of the acellular components of the micro-organ. Exemplary, but non-limiting methods for devitalization and removal of cells from micro-organs are detailed U.S. Pat. Nos. 7,297,540 and 10,093,896. In one exemplary method, micro-organs are treated with ammonium hydroxide and detergent (SDS) and washed thoroughly in saline to remove cellular mass. Alternatively, the micro-organs can be treated with 1-2 M NaCl and detergent (e.g. Triton, SDS, etc.). In another embodiment, micro-organs from cryopreserved tissue are washed repeatedly and extensively in cold water, or in 1 M NaCl followed by detergent solution, finally washed and stored in water with or without preservatives (e.g. antibiotics) before use. In yet another embodiment, the devitalized, acellular micro-organ matrices are stored frozen until use. Alternatively, the devitalized, acellular micro-organ matrices are dried (e.g. lyophilized), and rehydrated in water or medium before use.

The dimensions of the MOMs are selected to provide diffusion of adequate nutrients and gases such as oxygen to every cell seeded within the three-dimensional structure, as well as diffusion of cellular waste out of the MOM so as to minimize cellular toxicity and concomitant death due to localization of the waste in the MOM.

Typically, the dimensions of the devitalized, acellular, lung tissue-derived three-dimensional scaffold (MOM) according to the present invention are selected such that the point deepest within said scaffold is at least about 100 micrometers and not more than about 225 micrometers away from the nearest surface of the scaffold. Thus, when populated with cells, the cells positioned deepest within the scaffold are at least about 100 micrometers and not more than about 250 micrometers away from the cells positioned at a nearest surface formed on the scaffold.

Thus, in some embodiments, the acellular three-dimensional scaffold is of dimensions selected such that the point deepest within said scaffold is at least about 100 micrometers and not more than about 250 micrometers away from the nearest surface of the scaffold.

In one embodiment, the scaffolds are devitalized, acellular tissue sections in the range of 100-500 micrometers thick. In another embodiment, the scaffolds are devitalized, acellular tissue sections about 300 micrometers thick. In yet another embodiment, the scaffolds are devitalized, acellular tissue sections about 300 micrometers thick and 5-12 mm wide by 5-12 mm long, including each value within the range. In yet another embodiment, the scaffolds are devitalized, acellular tissue sections about 300 micrometers thick and at least 5, at least 10, at least 15, at least 20, at least 25, at least 30, at least 40, at least 50 mm in length, and at least 5, at least 10, at least 15, at least 20, at least 25, at least 30, at least 40, at least 50 mm in width. Each possibility represents a separate embodiment of the present invention.

The micro-organs suitable for preparation of the micro-organ matrices of the present invention can be prepared from a lung tissue derived from any animal, preferably a mammal. In some particular embodiments, the lung tissue is a porcine lung tissue. In some embodiments, the micro-organs are prepared from lungs excised under sterile conditions from freshly killed animals, kept on ice, rinsed with medium (e.g. Ringer or DMEM), and sectioned into 300 micrometer slices using a tissue chopper. In another embodiment, the micro-organs are prepared from fresh-frozen cryopreserved tissue or cryopreserved tissue sections, thawed to −2 to −20° C. for sectioning and sectioned, for example, using a pre-cooled tissue chopper or slicer.

Exemplary procedures for the preparation of devitalized, acellular, three-dimensional scaffolds are described in detail in U.S. Pat. No. 10,093,896. In brief, the procedure includes the following steps: First, lung-tissue derived micro-organs are prepared, e.g., from lungs of pigs. Adult animals are sacrificed and the lungs are removed under sterile conditions. The lungs are kept on ice, rinsed and cut into 300 μm slices using a tissue slicer to form MOs.

The MOs are decellularized and subsequently sterilized, for example, with 0.1% PAA for 30 min and washed 3 times with DDW 3×30 min each, prior to storage. For decellularization, the MOs may be treated with 0.67% ammonium hydroxide in 0.5% SDS. After all of the cellular mass is removed the remaining extracellular (devitalized, acellular) mass is washed thoroughly, e.g., in five changes of PBS, after which the matrix is ready for use as a three-dimensional scaffold. Cells can then be seeded onto the MOM. Optionally, the MOMs can be frozen at −20° C. until required, thawed and washed, e.g., 3 times in PBS and 2 times in culture medium, prior to using in culture.

Alternatively, decellularizing the MOs can be performed by immersing the MOs in one of (a) 10-50 mM NH₄OH+0.2-3% TritonX-100; (b) 1-2M NaCl; (c) 1-2M NaCl+0.2-3% Triton X-100; or (d) 1-2M NaCl+0.01-0.1% SDS, for a period of 45 min. The resulting devitalized acellular MOMs can then undergo washes, e.g., 5×15 min washes in sterile distilled H₂O. At this stage the resulting MOMs can be stored frozen at −80 C or rinsed, e.g., by 5×15 min washes in PBS, prior to using.

Alternatively, the organ (lung) is excised fresh, washed in water, cleaned and optionally stored on ice for up to 1.5 hours. Prior to slicing, the organ is cut into columns 12 mm×12×20-40 mm and frozen at −20° C. to −80° C. and kept frozen until required for MOM preparation. Prior to cutting the organ columns are equilibrated to −2° to −10° C. and then sliced into 200 to 500 μm×12×12 mm sections using a pre-cooled tissue slicer.

MOMs can then be prepared from the cut sections as follows:

a. Cut sections are washed in cold sterile DDW for a 1 hour, with water changes every 15 minutes (approximately 50 ml of DDW for each wash);

b. Cut sections are washed in room temperature (r.t.) sterile DDW for an additional 4 hours, with water changes every 20 minutes (approximately 50 ml of DDW for each wash); and

c. Cut sections are stored at −80° C. in minimal volume of DDW until required for seeding.

Or alternatively:

a. Cut sections are washed in room temperature sterile DDW for additional 4 hours, with water changes every 20 minutes (approximately 50 ml of DDW for each wash); and

b. Cut sections are kept overnight at 4° in DDW.

C. Cut sections are then stored at −80° C. in minimal volume of DDW until required for seeding.

Or alternatively:

a. Cut sections are washed in room temperature sterile DDW for additional 4 hours, with water changes every 20 minutes (approximately 50 ml of DDW for each wash); and

b. Cut sections are kept overnight at 4° in DDW.

C. Cut sections are stored in PBS in 10× antibiotic for 1 to 10 days prior to seeding cells on the resulting MOMs.

Or alternatively:

a. Cut sections are placed in 1M NaCl for 1 hour, with 3 changes, every 20 min;

b. Cut sections are transferred to a solution of 0.5% Triton in DDW for 3 hours and changed every 30 minutes;

c. Cut sections are washed with DDW 3×15 min each; and

d. Cut sections are stored in PBS in 10× antibiotic for 1 to 10 days prior to seeding cells on the resulting MOMs.

Or alternatively:

a. Cut sections are placed in 1M NaCl for 1 hour, with 3 changes, every 20 min;

b. Cut sections are transferred to a solution of 0.5% Triton in H₂O for 3 hours and changed every 30 minutes;

c. Cut sections are washed with H₂O 5×15 min each: and

d. Cut sections are then stored at −80° C. in minimal volume of DDW until required for seeding.

Methods of seeding cells on scaffolds are known in the art. Cells can be seeded on the scaffold by static loading, by seeding in stirred flask bioreactors, in a rotating wall vessel, or using direct perfusion of the cells in medium in a bioreactor. The cells may be seeded directly onto the micro-organ matrix scaffold. The cells in their medium may be absorbed onto the interior and exterior surfaces of the scaffold.

Cells can be seeded at different densities. In some embodiments, cells are seeded at about 1×10⁴ to about 1×10⁶ cells per micro-organ matrix. In additional embodiments, cells are seeded at 2×10⁵ to about 1×10⁶ cells per 2-4 micro-organ matrices. In additional embodiments, cells are seeded at 2×10⁵ to about 1×10⁶ cells per 5-7 micro-organ matrices.

In some embodiments, a plurality of MOMs are cultured in a single cell culture vessel.

The MOM-cell cultures may be maintained in any suitable culture vessel such as 12-well microplates and may be maintained at 37° C. in 5% CO₂.

Cell Populations and Differentiation Procedures

Cell differentiation is the process by which an unspecialized (uncommitted) or less specialized cell acquires the features of a specialized cell.

As used herein, the lineage of a cell defines to what cells it can give rise.

In vitro differentiation of pluripotent stem cells into insulin-secreting beta cells follows a sequence of developmental stages that mimics pancreatic organogenesis, starting with the differentiation to definitive endoderm (DE), followed by sequential differentiation through several stages, referred to as “stepwise differentiation”, in which a plurality of differentiation factors are sequentially applied until beta cells are obtained. At each stage a medium containing suitable differentiation factors is added, which induce differentiation of the cells to the next stage, after which the medium is replaced with a medium containing the factors needed for differentiating the cells to the next stage, and so forth. Each stage is characterized by one or more markers expressed by the cells.

The term “differentiation factor” as used herein refers to a molecule, such as a small molecule, protein or peptide, that induces cells to differentiate to a desired cell type. For example, a “definitive endoderm differentiation factor” refers to a differentiation factor that induces differentiation to definitive endoderm cells. A “primitive gut differentiation factor” refers to a differentiation factor that induces differentiation to primitive gut cells, and so forth.

“Markers”, as used herein, are nucleic acid or polypeptide molecules that are differentially expressed in a cell of interest. In this context, differential expression means an increased level for a positive marker and a decreased level for a negative marker as compared to a cell of a different developmental stage. The detectable level of the marker nucleic acid or polypeptide is sufficiently higher or lower in the cells of interest compared to cells of a different developmental stage, such that the cells of interest can be identified and distinguished from the other cells using any of a variety of methods known in the art.

As used herein, a cell is “positive” for a specific marker when the specific marker is detected in the cell. The cell is “negative” for a specific marker when the specific marker is not detected in the cell. The expression of a certain marker by a population of cells may be determined qualitatively, for example using immunostaining techniques, or quantitatively, for example using FACS, where the percentage of cells within the population that express the marker can be determined.

The cells according to the present invention are typically human cells.

“Pluripotent stem cells” are stem cells with the potential to differentiate into cells of all three germinal layers: endoderm, mesoderm and ectoderm tissues. Characteristic markers of pluripotent stem cells include one or more of the following: Oct4, Nanog, Sox2, Klf4, c-myc, CDH1. Additional markers characteristic of pluripotent stem cells include ABCG2, cripto, FOXD3, CONNEXIN43, CONNEXIN45, hTERT, UTF1, ZFP42 (Rex1), SSEA-3, SSEA-4, Tra 1-60, Tra 1-81. Characteristic markers of pluripotent stem cells are listed, for example, in: www.rndsystems.com/research-area/embryonic-and-induced-pluripotent-stem-cell-markers.

Pluripotent stem cells may be readily expanded in culture using various feeder layers or by using matrix protein coated vessels. The vessels may be coated by extracellular matrix components, such as those derived from basement membrane or that may form part of adhesion molecule receptor-ligand couplings. For example, reconstituted basement membrane sold under the trademark Matrigel™ may be used. Matrigel™ is a soluble preparation from Engelbreth-Holm Swarm tumor cells that gels at room temperature to form a reconstituted basement membrane. Other extracellular matrix components and component mixtures known in the art are suitable as an alternative.

The pluripotent stem cells may be plated onto the substrate in a suitable distribution and in the presence of a medium, which promotes cell survival, propagation, and retention of the desirable characteristics. Suitable culture media include, for example, feeder-free, serum-free and complete cell culture medium such as mTeSR™. Pluripotent cells may be readily removed from culture plates using enzymatic, mechanical or use of various calcium chelators such as EDTA (Ethylenediaminetetraacetic acid). Alternatively, pluripotent cells may be expanded in suspension in the absence of any matrix proteins or a feeder layer.

In some embodiments, the pluripotent stem cells are embryonic stem cells. In other embodiments, the pluripotent stem cells are other than embryonic stem cells. In additional embodiments, the pluripotent stem cells are induced pluripotent stem cells.

The types of pluripotent stem cells that may be used include established lines of pluripotent cells derived from tissue formed after gestation, including pre-embryonic tissue (such as, for example, a blastocyst), embryonic tissue, or fetal tissue taken any time during gestation, typically but not necessarily, before approximately 10 to 12 weeks gestation. Non-limiting examples are established lines of human embryonic stem cells (hESCs) or human embryonic germ cells, such as, for example the human embryonic stem cell lines HES-2, H1, H7, and H9. Also suitable are cells taken from a pluripotent stem cell population already cultured in the absence of feeder cells. The human embryonic cells are preferably prepared without the destruction of a human embryo, as described for example in Chung et al., 2008, Cell Stem Cell., 2(2):113-7.

Also suitable are induced pluripotent stem cells (iPSCs) or reprogrammed pluripotent cells that can be derived from adult somatic cells using forced expression of a number of pluripotent related transcription factors, such as OCT4, NANOG, Sox2, KLF4, and ZFP42 (Loh et al., Annu Rev Genomics Hum Genet, 2011, 12:165-185). The cells can be derived from either autologous sources or from allogeneic sources.

As pluripotent stem cells differentiate towards functional beta cells, they differentiate through various stages each of which may be characterized by the presence or absence of particular markers. Differentiation of the cells into these stages is achieved by the specific culturing conditions including the presence and lack of certain factors added to the culture media. Suitable growth media include chemically defined media containing sufficient quantities of vitamins, minerals, salts, glucose and amino acids. Examples are given below.

In some embodiments, the differentiation from pluripotent stem cells to beta cells comprises: differentiating the pluripotent stem cells to definitive endoderm cells; differentiating the definitive endoderm cells to primitive gut cells; differentiating the primitive gut cells to posterior foregut cells; differentiating the posterior foregut cells to pancreatic endoderm cells; differentiating the pancreatic endoderm cells to endocrine precursor cells (also called pancreatic endocrine progenitor cells); and differentiating the endocrine precursor cells to beta cells.

In some embodiments, the differentiation from pluripotent stem cells to beta cells comprises: differentiating the pluripotent stem cells to definitive endoderm cells; differentiating the definitive endoderm cells to primitive gut cells; differentiating the primitive gut cells to posterior foregut cells; differentiating the posterior foregut cells to pancreatic progenitor 1 cells, differentiating the pancreatic progenitor 1 cells to pancreatic progenitor 2 cells, differentiating the pancreatic progenitor 2 to endocrine precursor cells (also called pancreatic endocrine progenitor cells); and differentiating the endocrine precursor cells to beta cells.

“Progenitor cells” as used herein refers to undifferentiated cells that have a cellular phenotype that is more primitive (e.g., is at an earlier step along a developmental pathway or progression than is a fully differentiated cell) relative to a cell to which it can give rise by differentiation. Some progenitor cells can produce progeny that are capable of differentiating into more than one cell type. A progenitor cell is thought to be committed to a particular path of differentiation and will, under appropriate conditions, eventually differentiate along this pathway. The progenitor cells according to the present invention are progenitor cells of the pancreatic lineage. In some embodiments, the progenitor cells of the pancreatic lineage are selected from the group consisting of: definitive endoderm cells, primitive gut cells, posterior foregut cells, pancreatic endoderm cells and endocrine precursor cells (also called pancreatic endocrine progenitor cells). In additional embodiments, the progenitor cells of the pancreatic lineage according to the present invention are selected from the group consisting of: definitive endoderm cells, primitive gut cells, posterior foregut cells, pancreatic progenitor 1 cells, pancreatic progenitor 2 cells and endocrine precursor cells (also called pancreatic endocrine progenitor cells).

“Definitive endoderm cells” (DE cells) are cells which form the gastrointestinal tract and its derivatives e.g. pancreas or liver. Characteristic markers of definitive endoderm cells include one or more of the following markers: CXCR4, c-kit, PDX1, FoxA2, GP2, Sox17, and GSC. Definitive endoderm cells according to the present invention express one or more, preferably two or more, more preferably three or more, even more preferably all of the aforementioned markers. In some embodiments, the definitive endoderm cells according to the present invention express markers comprising CXCR4 and c-kit. Additional characteristic markers of definitive endoderm cells include HNF3 beta, GATA4, Cerberus, OTX2, goosecoid, CD99, and MIXL1. Characteristic markers of definitive endoderm cells are listed, for example, in: www.rndsystems.com/research-area/early-endodermal-lineage-markers.

The differentiation from pluripotent stem cells into definitive endoderm cells can be carried out by plating the pluripotent stem cells on a tissue culture substrate coated with an extracellular matrix, and culturing the pluripotent stem cells in a chemically defined complete, serum- and animal component-free, medium comprising activin A, to obtain definitive endoderm cells. Exemplary procedures are described in the Examples section below.

In some embodiments, the differentiation from pluripotent stem cells into definitive endoderm cells may be carried out by a process comprising:

(i) plating the pluripotent stem cells on a low-attachment tissue culture substrate and culturing the pluripotent stem cells in a chemically defined complete, serum-free, medium comprising BMP4 to obtain embryoid bodies;

(ii) collecting the embryoid bodies and culturing them in a chemically defined complete, serum-free, medium comprising BMP4, bFGF and activin A; and

(iii) collecting the embryoid bodies from step (ii) and culturing them in a chemically defined complete, serum-free, medium comprising VEGF, activin A and bFGF to obtain definitive endoderm cells.

In some embodiments, the differentiation from pluripotent stem cells into definitive endoderm cells is carried out by a process comprising:

(i) plating the pluripotent stem cells on a low-attachment tissue culture substrate and culturing the pluripotent stem cells in a chemically defined complete, serum-free, medium comprising glutamine, ascorbic acid, monothioglycerol (MTG) and BMP4 for 24 hours to obtain embryoid bodies;

(ii) collecting the embryoid bodies and culturing them in a chemically defined complete, serum-free, medium comprising glutamine, ascorbic acid, monothioglycerol (MTG), BMP4, bFGF and activin A for 48-72 hours; and

(iii) collecting the embryoid bodies from step (ii) and culturing them in a chemically defined complete, serum-free, medium comprising glutamine, ascorbic acid, monothioglycerol (MTG), VEGF, activin A and bFGF for at least 24 hours to obtain definitive endoderm cells.

“Primitive gut cells” (PG cells), also termed “primitive gut tube cells” or “gut tube cells”, are cells derived from definitive endoderm that express characteristic markers comprising one or more of the following markers: FoxA1, HNF1-beta (HNF1B), HNF4-alpha (HNF4A). Primitive gut cells according to the present invention express one or more, preferably two or more, more preferably all of the aforementioned markers. An additional characteristic marker of primitive gut cells is HNF3-beta (FOXA2). Primitive gut cells can give rise to endodermal organs, such as liver, pancreas, stomach, and intestine.

The differentiation from definitive endoderm cells into primitive gut cells can be carried out by culturing in a chemically defined medium which may be supplemented with supplements such as ITS-X, GlutaMAX™ and/or B27 and comprising one or more primitive gut differentiation factor. Examples of primitive gut differentiation factors include KGF, FGF7, and vitamin C. Each possibility represents a separate embodiment of the present invention. In some exemplary embodiments, the primitive gut differentiation factor is KGF.

“Posterior foregut cells” (PFG cells), also termed “posterior gut tube cells”, are cells that express characteristic markers comprising one or more of the following markers: PDX1, HNF6, SOX9, PROX1. Posterior foregut cells can give rise to posterior stomach, pancreas, liver, and a portion of the duodenum.

The differentiation from primitive gut cells into posterior foregut cells can be carried out by culturing in a chemically defined medium which may be supplemented with supplements such as ITS-X, GlutaMAX™ and/or B27 and comprising one or more posterior foregut differentiation factor. Examples of combinations of posterior foregut differentiation factors include KGF+SANT-1+ retinoic acid (RA)+ LDN-193189+ PdBU; KAAD-cyclopamine+ retinoic acid (RA)+ LDN-193189; and FGF7+vitamin C+ TPB+SANT. Each possibility represents a separate embodiment of the present invention. In some exemplary embodiments, the posterior foregut differentiation factors are KGF+SANT-1+ retinoic acid (RA)+ LDN-193189+ PdBU.

In some embodiments, the primitive gut cells are differentiated into pancreatic endoderm cells, subsequently to endocrine precursor cells and finally to beta cells. Such a procedure is exemplified in Example 2 hereinbelow. A person skilled in the art would appreciate that an alternative pathway may be applied, which involves two sub-stages, as follows: the primitive gut cells are first differentiated into pancreatic progenitor 1 cells (PP1 cells), next into pancreatic progenitor 2″ cells (PP2 cells), and subsequently into endocrine precursor cells and finally to beta cells. An exemplary protocol is provided in Example 1 hereinbelow.

“Pancreatic endoderm cells” are an intermediate cell population in the development of the pancreatic lineage. Characteristic markers of pancreatic endoderm cells include one or more of the following markers: NKx6.1 and PDX1. Pancreatic endoderm cells according to the present invention express one or both markers.

The differentiation from posterior foregut cells into pancreatic endoderm cells can be carried out by culturing in a chemically defined medium which may be supplemented with supplements such as ITS-X and comprising one or more pancreatic endoderm differentiation factor. An exemplary combination of pancreatic endoderm differentiation factors is KGF+SANT-1+RA+ iBET151.

“Pancreatic progenitor 1 cells (PP1 cells)” are another intermediate cell population in the development of the pancreatic lineage. Characteristic markers of PP1 cells include one or more of the following markers: PDX1, NKX6.1, HNF6, Prox1, Sox9, NEUROD1. PP1 cells according to the present invention express one or more, preferably two or more, more preferably three or more, even more preferably all of the aforementioned markers. Additional characteristic markers of PP1 cells include FOXA2, CDX2, SOX9, and HNF4α. Pancreatic progenitor 1 cells are characterized by co-expression of PDX1, FOXA2, HNF6 and NKX6-1, where the expression of PDX1 is increased compared to gut tube cells.

The differentiation from posterior foregut cells into PP1 cells can be carried out by culturing in a chemically defined medium which may be supplemented with supplements such as GlutaMAX™ and B27 and comprising one or more PP1 differentiation factor. Examples of combinations of PP1 differentiation factors include EGF+FGF7, KGF+ retinoic acid (RA)+SANT1+Y-27632+ LDN-193189+ PdbU, and RA+cyclopamine+Noggin, iBET and ITS. Each possibility represents a separate embodiment of the present invention.

“Endocrine pancreatic progenitor cells”, also termed “pancreatic progenitor 2” cells (PP2 cells)”, are another intermediate cell population in the development of the pancreatic lineage. Characteristic markers of PP2 cells include one or more of the following markers: NKX6.1, PTF1A, NGN3, NKX2.2. PP2 cells according to the present invention express one or more, preferably two or more, more preferably three or more, even more preferably all of the aforementioned markers. Additional characteristic markers of PP2 cells include PDX1.

The differentiation from PP1 cells into PP2 cells can be carried out by culturing in a chemically defined medium which may be supplemented with supplements such as GlutaMAX™ and B27 and comprising one or more PP2 differentiation factor. Examples of combinations of PP2 differentiation factors include an ALK-5 inhibitor+heparin+FGF7+Y-27632, KGF+ retinoic acid+SANT1+Y-27632+Activin A, iBET and ITS. Each possibility represents a separate embodiment of the present invention.

“Endocrine precursor cells”, also called “pancreatic endocrine progenitor cells”, and “endocrine progenitor cells”, (abbreviated EN cells), refers to pancreatic endoderm cells capable of becoming a pancreatic hormone expressing cell. Characteristic markers of endocrine precursor cells include one or more of the following markers: PDX1, GP2, Nkx6.1, INS, CHGA, GCG, and SST. Endocrine precursor cells according to the present invention express one or more, preferably two or more, more preferably three or more, even more preferably all of the aforementioned markers. Additional characteristic markers of endocrine precursor cells include NGN3. NKX2.2, NeuroD1, ISL1, PAX4, PAX6, ARX.

Differentiation from pancreatic endoderm cells into endocrine precursor cells can be carried out by culturing in a chemically defined medium which may be supplemented with supplements such as ITS-X and comprising one or more endocrine precursor differentiation factor. An exemplary combination of endocrine precursor differentiation factors is SANT-1+RA+PI 3-K Inhibitor XXI+ Alk5 Inhibitor II (Alk5i II)+ Triiodothyronine (T3)+ Betacellulin.

Differentiation from PP2 cells into endocrine precursor cells can be carried out by culturing in a chemically defined medium which may be supplemented with supplements such as GlutaMAX™ and B27 and comprising one or more endocrine precursor differentiation factor. Examples of combinations of endocrine precursor differentiation factors include an ALK5 inhibitor+zinc sulfate+heparin+a gamma secretase inhibitor+Y-27632, retinoic acid+SANT1+T3+XX1+an ALK5 inhibitor+heparin+betacellulin. Each possibility represents a separate embodiment of the present invention.

“Beta cells” (“β cells”) are pancreatic endocrine cells capable of expressing insulin, but not glucagon, somatostatin, ghrelin, and pancreatic polypeptide. Cells expressing markers characteristic of beta cells can be characterized by the expression of insulin (INS) and at least one of the following markers: PDX1, GP2, NKX6.1, C-peptide and MAFA. Beta cells according to the present invention express one or more, preferably two or more, more preferably three or more, even more preferably all of the aforementioned markers. The beta cells may be further characterized by being negative for GCG, PC1/3, SST and CHGA.

In some embodiments, the beta cells obtained by the methods of the present invention comprise at least 20% MAFA+ monohormonal cells at the end of the differentiation process, for example at least 30%, at least 40%, at least 50%, at least 60% MAFA+ monohormonal cells at the end of the differentiation process. Each possibility represents a separate embodiment of the present invention.

In some embodiments, the beta cells obtained by the methods of the present invention comprise at least 10% NKX6-1+/C-peptide+ monohormonal cells at the end of the differentiation process. In additional embodiments, the beta cells obtained by the methods of the present invention comprise at least 20%, at least 30%, at least 40%, at least 50%, at least 60% NKX6-1+/C-peptide+ monohormonal cells at the end of the differentiation process. Each possibility represents a separate embodiment of the present invention.

In some embodiments, at least 10% (for example, at least 20%, at least 30%, at least 40%, at least 50%, at least 60%) of the cells in the beta cell population obtained by the methods of the present invention are positive for INS, PDX1 and NKX6.1, and negative for GCG, PC1/3, SST and CHGA. Additional characteristic markers include NKX2.2, NeuroD1, ISL1, GLUT2, and PAX6.

Beta cells are also characterized by glucose-responsive insulin secretion. particularly, beta cells are characterized by a bi-phasic insulin secretion in response to glucose stimulation. In some embodiments, at least 10% (for example, at least 20%, at least 30%, at least 40%, at least 50%, at least 60%) of the cells in the beta cell population obtained by the methods of the present invention are insulin-positive, preferably secrete insulin in a bi-phasic glucose-responsive manner, as tested and exemplified hereinbelow.

The differentiation from endocrine precursor cells (pancreatic endocrine progenitor cells) into beta cells can be carried out by culturing in a chemically defined medium which may be supplemented with supplements such as GlutaMAX™ and B27 and comprising one or more beta cell differentiation factor. Examples of beta cell differentiation media include CMRL or RPMI plus GlutaMAX™, supplemented with 10% FBS, 1% B27 and 1% penicillin streptomycin, with the addition of differentiation factors such as Y-27632, T3+an ALK5 inhibitor (e.g., 10 μM Alk5i II+1 μM T3), an ALK5 inhibitor+T3+N-Cys+AXL inhibitor. Each possibility represents a separate embodiment of the present invention. An additional example of a beta cell differentiation medium is CMRL supplemented with 10% FBS with the addition of the differentiation factors Alk5i II, L-3,30,5-Triiodothyronine (T3) and nicotinamide.

“Insulin-producing beta cells” or “insulin-producing cells” according to the present invention are functional beta-cells exhibiting glucose-stimulated insulin secretion (“GSIS”).

The efficiency of differentiation may be determined by exposing a cell population to an agent, such as an antibody, that specifically recognizes a protein marker expressed by the differentiated cells of interest. Methods for assessing expression of protein and nucleic acid markers in cultured or isolated cells are standard in the art. These methods include RT-PCR, qRT-PCR, microarray, Northern blots, in situ hybridization and immunoassays such as immunocytochemical analysis, western blotting and for markers that are accessible in intact cells, flow cytometry analysis (FACS).

In some embodiments, the methods of the present invention comprise seeding progenitor cells of the pancreatic lineage selected from the group consisting of definitive endoderm cells, primitive gut cells, posterior foregut cells, pancreatic endoderm cells and endocrine precursor cells on a devitalized, acellular, lung tissue-derived three-dimensional scaffold. Each possibility of progenitor cells to be seeded on the scaffold is a separate embodiment of the present invention.

In some embodiments, the methods of the present invention comprise seeding progenitor cells of the pancreatic lineage selected from the group consisting of definitive endoderm cells, primitive gut cells, posterior foregut cells, pancreatic progenitor 1 (PP1) cells, endocrine pancreatic progenitor (PP2) cells and pancreatic endocrine progenitor cells on a devitalized, acellular, lung tissue-derived three-dimensional scaffold. Each possibility of progenitor cells to be seeded on the scaffold is a separate embodiment of the present invention.

In some embodiments, the methods of the present invention comprise seeding progenitor cells of the pancreatic lineage which are at least at the pancreatic endoderm stage. According to these embodiments, the progenitor cells of the pancreatic lineage may be selected from pancreatic endoderm cells and endocrine precursor cells and any stage in between. In some particular embodiments, the progenitor cells of the pancreatic lineage are pancreatic endoderm cells.

The differentiation of pluripotent stem cells to beta cells according to the present invention begins in a 2D culture and continues on a 3D scaffold as described herein.

In some embodiments, a differentiation method according to the present invention comprises: differentiating pluripotent stem cells into pancreatic endoderm cells by a stepwise differentiation in a 2D cell culture; changing the medium in the 2D cell culture to a medium comprising one or more endocrine precursor differentiation factors and incubating the cells for 1 day; seeding the cells after the 1 day of incubation in the medium comprising one or more endocrine precursor differentiation factors on a scaffold as described herein in a culture medium comprising one or more endocrine precursor differentiation factors, to obtain endocrine precursor cells on the scaffold; and culturing the scaffold with the obtained endocrine precursor cells in a medium comprising one or more beta cell differentiation factor, to obtain insulin-producing beta cells on the scaffold.

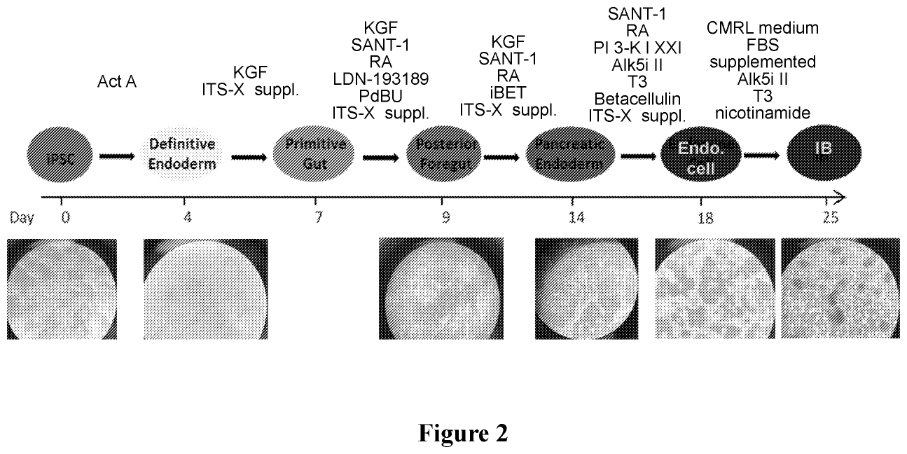

In some embodiments, the differentiation from pluripotent stem cells into insulin-producing beta cells is carried out according to a 25-day protocol as detailed in Example 2 hereinbelow and illustrated in FIG. 2. In some embodiments, the progenitor cells of the pancreatic lineage seeded on the scaffold according to the present invention are any of Day 4-Day 18 cells. In some particular embodiments, the progenitor cells of the pancreatic lineage seeded on the scaffold according to the present invention are Day 14-Day 18 cells, meaning that the cells were grown until Day 14, Day 15, Day 16, Day 17 or Day 18 in a 2D cell culture, and subsequently seeded on the scaffold and continued the differentiation process on the scaffold. Each possibility represents a separate embodiment of the present invention. In some exemplary embodiments, the progenitor cells of the pancreatic lineage seeded on the scaffold are Day 15 cells according to the differentiation process detailed in Example 2 and illustrated in FIG. 2.

In some embodiments, a method for generating a population of insulin-producing beta cells is provided, the method comprising:

(i) culturing a devitalized, acellular, lung tissue-derived three-dimensional scaffold seeded with pancreatic endoderm cells in a culture medium comprising one or more endocrine precursor differentiation factor, to obtain endocrine precursor cells on the scaffold; and

(ii) replacing the medium of step (i) with a medium comprising one or more beta cell differentiation factor, to obtain beta cells on the scaffold.

In some embodiments, a method for generating a population of insulin-producing beta cells is provided, the method comprising:

(i) culturing a devitalized, acellular, lung tissue-derived three-dimensional scaffold seeded with definitive endoderm cells in a culture medium comprising one or more primitive gut tube differentiation factor, to obtain primitive gut cells on the scaffold;

(ii) replacing the medium of step (i) with a medium comprising one or more posterior foregut differentiation factor, to obtain posterior foregut cells on the scaffold;

(iii) replacing the medium of step (ii) with a medium comprising one or more PP1 differentiation factor, to obtain PP1 cells on the scaffold;

(iv) replacing the medium of step (iii) with a medium comprising one or more PP2 differentiation factor, to obtain PP2 cells on the scaffold;

(v) replacing the medium of step (iv) with a medium comprising one or more pancreatic endocrine progenitor differentiation factor, to obtain pancreatic endocrine progenitor cells on the scaffold; and
(vi) replacing the medium of step (v) with a medium comprising one or more beta cell differentiation factor, to obtain beta cells on the scaffold.

In some embodiments, the methods of the present invention further comprise differentiating pluripotent stem cells to the progenitor cells prior to seeding on the scaffold.

In some embodiments, the progenitor cells are definitive endoderm cells, and the sequentially applying a plurality of differentiation factors comprises:

(i) culturing the scaffold seeded with the definitive endoderm cells in a medium comprising FGF7, to obtain primitive gut tube cells;

(ii) culturing the scaffold with the primitive gut tube cells in a medium comprising KAAD-cyclopamine, retinoic acid and LDN 193189, to obtain posterior foregut cells;

(iii) culturing the scaffold with the posterior foregut cells in a medium comprising EGF and FGF7, to obtain PP1 cells;

(iv) culturing the scaffold with the PP1 cells in a medium comprising ALK5 inhibitor, heparin, FGF7 and Y-27632, to obtain PP2 cells;

(v) culturing the scaffold with the PP2 cells in a medium comprising T3, ALK5 inhibitor, zinc sulfate, heparin, gamma secretase inhibitor and Y-27632, to obtain pancreatic endocrine progenitor cells; and

(iv) culturing the scaffold with the pancreatic endocrine progenitor cells in a medium comprising FBS and Y-27632, to obtain beta cells.

Exemplary procedures for stepwise differentiation and generation of beta cells according to the present invention is detailed in the Examples section below. Alternative procedures may be used, which employ different differentiation factors at each stage. For example, the following differentiation process may be carried out:
differentiating pluripotent stem cells to definitive endoderm (DE) cells by culturing in a medium comprising Activin A and CHIR-99021;
differentiating DE cells to primitive gut (PG) cells by culturing in a medium comprising KGF;
differentiating PG cells to posterior foregut (PFG) cells by culturing in a medium comprising FGF7;
differentiating PFG cells to pancreatic progenitor (PP1) cells by culturing in a medium comprising KGF, retinoic acid, SANT1, Y-27632, LDN-193189 and PdbU;
differentiating PP1 cells to endocrine pancreatic progenitor (PP2) by culturing in a medium comprising KGF, retinoic acid, SANT1, Y-27632 and Activin A;
differentiating PP2 cells to pancreatic endocrine progenitor (EN) cells by culturing in a medium comprising retinoic acid, SANT1, T3, XX1, Alk-5 inhibitor, heparin and betacellulin; and
differentiating EN cells to beta cells by culturing in a medium comprising T3, Alk-5 inhibitor and CMRL.

In some embodiments, in addition to the above-described media and differentiation factors, at least one type of supporting cells are added to the MOMs, in order to support the differentiation and survival of the differentiating cells and subsequently the obtained beta cells. Supporting cells disclosed herein include at least one of endothelial cells and mesenchymal stem cells. In some embodiments, both endothelial cells and mesenchymal stem cells are seeded on the MOMs as supporting cells.

Endothelial cells such as HUVEC, endothelial cells from pancreas, liver, etc. may be used, for example, at a density of 5000 cells/MOM.

Mesenchymal stem cells (MSCs) such as MSCs from bone marrow, adipose tissue, placenta and Wharton Jelly (umbilical cord) may be used, for example, at a density of 10000 cells/MOM.

Artificial Micro-Organs

As used herein, the term “artificial micro-organ” or “engineered micro-organ” refers to a micro-organ scaffold with beta cells differentiated according to the present invention cultured thereon, having beta cell-specific functions when cultured and, optionally organized in a micro-organ-like three-dimensional tissue structure.

In some embodiments, the artificial micro-organ comprises cells expressing at least one cell-specific protein after at least 7 days in culture. In other embodiments, the cells express the at least one cell-specific protein after at least 10, at least 15, at least 20, at least 30, at least 50 and alternatively at least 70 days in culture.

In some embodiments, characteristic beta cell functions include, but is not limited to, expression of Pdx1 and insulin and glucose-responsive insulin secretion. Methods for monitoring beta cell specific protein expression include, but are not limited to RT-PCR for transcription of relevant genes, immunohistochemistry and quantitative immunodetection techniques such as ELISA.

Glucose responsive insulin secretion can be determined by the change in insulin secretion of beta cell-MOM cultures when the concentration of glucose in the medium is raised from “low glucose” to “high glucose” levels, as described, for example, by Marchetti et al (Diabetes, 1994; 43:827-30). Such a protocol, using 3 mM glucose as the low levels, and 16.7 mM glucose as the high levels, is currently the standard procedure for testing beta cell function before transplantation.

In some embodiments, the beta cells of the artificial micro-organ of the present invention are characterized by glucose responsive insulin secretion after at least 7 days in culture, alternatively at least 10 days in culture, alternatively at least 14 days in culture, alternatively at least 20 days in culture, alternatively at least 24 days in culture, alternatively at least 28 days in culture, alternatively at least 35 days in culture, alternatively at least 40 days in culture, alternatively at least 50 days in culture, alternatively at least 60 days in culture, alternatively at least 70 days in culture and alternatively at least 75 days in culture. Each possibility represents a separate embodiment of the present invention. In some other embodiments, the beta cells express Pdx1 following at least 7 days in culture, alternatively at least 14 days in culture, alternatively at least 20 days in culture, alternatively at least 28 days in culture and alternatively at least 35 days in culture. Each possibility represents a separate embodiment of the present invention.

In some embodiments, a population of beta cells obtained by the differentiation procedure according to the present invention comprises an increased percentage of cells that produce insulin in a glucose-responsive manner compared to a population differentiated in a 2D culture.

In some embodiments, a population of beta cells obtained by the differentiation procedure according to the present invention secretes insulin in response to glucose at higher level compared to a population differentiated in a 2D culture and/or have a better glucose stimulation index, as calculated by the ratio of insulin secreted in high glucose to low glucose.

In some embodiments, a population of beta cells obtained by the differentiation procedure according to the present invention exhibits improved insulin secretion kinetics compared to a population differentiated in a 2D culture, determined for example by glucose perifusion or stimulation assays with other insulin secretagogues.

As used herein, a “2D culture” (two-dimensional culture) refers to differentiation in a vessel such as a plate, including a well coated with extracellular matrix.

Therapeutic Use

As used herein “diabetes” refers to a disease resulting either from an absolute deficiency of insulin (type 1 diabetes) due to a defect in the biosynthesis or production of insulin, or a relative deficiency of insulin in the presence of insulin resistance (type 2 diabetes), i.e., impaired insulin action, in an organism, typically a human. The diabetic patient thus has absolute or relative insulin deficiency, and displays, among other symptoms and signs, elevated blood glucose concentration, presence of glucose in the urine and excessive discharge of urine.

In some particular embodiments, the subject is afflicted with type 1 diabetes.

In additional particular embodiments, the diabetes is type 2 diabetes.

In some embodiments, the diabetes is diabetes caused by pancreatitis. Pancreatitis is a condition in which the pancreas becomes inflamed. Damage to insulin-producing cells in the pancreas from chronic pancreatitis can lead to diabetes.

In some embodiments, the diabetes is caused by pancreas inflammation or other causes to pancreas disfunction.

The subject is typically a human subject.

The term “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

As used herein, “transplanting” refers to providing the artificial micro-organ of the present invention to a location in a recipient's body. For example, the artificial micro-organ may be transplanted subcutaneously (SC) or via intraperitoneal (IP) injection.

It will be appreciated that more than one micro-organ can be transplanted at the same time to the same individual. Dosage and character of the micro-organs for transplantation are typically determined according to the patient's body weight and disease status, for example, according to the severity of the insulin deficiency.

In one embodiment, the micro-organs are transplanted within the subject soon after the differentiation to beta cells is completed. Alternatively, the differentiated cells can be cultured for hours, days or weeks before transplantation.

According to an aspect of the present invention there is provided a method of treating diabetes in a subject, the method comprising transplanting a therapeutically effective amount of an artificial micro-organ comprising beta cells differentiated on a devitalized, acellular lung tissue-derived matrix according to the present invention into the subject, thereby treating diabetes.

The artificial micro-organs may be transplanted to a human subject per se, or in a pharmaceutical composition where it is mixed with suitable carriers or excipients.

As used herein a “pharmaceutical composition” refers to a preparation comprising the artificial micro-organ described herein with other chemical components such as physiologically suitable carriers and excipients. The purpose of a pharmaceutical composition is to facilitate administration of the artificial micro-organ to a subject in need thereof.

The phrases “physiologically acceptable carrier” and “pharmaceutically acceptable carrier” which may be interchangeably used refer to a carrier or a diluent that does not cause significant irritation to the subject and does not abrogate the biological activity and properties of the artificial micro-organ.

The term “excipient” refers to an inert substance added to a pharmaceutical composition to further facilitate administration of the artificial micro-organ.

Pharmaceutical compositions may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries. Proper formulation is dependent upon the route of administration chosen.

For injection, the artificial micro-organs may be formulated in aqueous solutions, preferably in physiologically compatible buffers such as Hank's solution, Ringer's solution, or physiological salt buffer.

Pharmaceutical compositions suitable for use in context of the present invention include compositions wherein the active ingredients are contained in an amount effective to achieve the intended purpose. More specifically, a therapeutically effective amount means an amount of active ingredients (engineered micro-organs) effective to prevent, alleviate or ameliorate symptoms of the disorder (e.g. diabetes).

Determination of a therapeutically effective amount is within the capability of those skilled in the art.

Kits

In some embodiments, there is provided herein a kit for generating insulin-producing beta cells, comprising:

(i) a devitalized, acellular, lung tissue-derived three-dimensional scaffold;

(ii) a plurality of differentiation factors and optionally supporting cells (endothelial cells and MSCs) for conducting a stepwise differentiation of progenitor cells of the pancreatic lineage to beta cells; and

(iv) an instruction manual comprising instructions for seeding progenitor cells of the pancreatic lineage on the scaffold and for carrying out a stepwise differentiation on the scaffold such that cells remain on said scaffold throughout the differentiation process.

In additional embodiments, there is provided herein a kit for generating insulin-producing beta cells, comprising:
(i) a devitalized, acellular, lung tissue-derived three-dimensional scaffold seeded with progenitor cells of the pancreatic lineage;
(ii) optionally, endothelial cells and MSCs seeded onto the lung tissue-derived three-dimensional scaffold that support the differentiation, production and survival of the differentiating cells and subsequently the obtained beta cells;
(iii) a plurality of differentiation factors for conducting a stepwise differentiation of the progenitor cells of the pancreatic lineage to beta cells; and
(iv) an instruction manual specifying instructions for carrying out a stepwise differentiation on the scaffold such that cells remain on said scaffold throughout the differentiation process.

In some embodiments, the progenitor cells of the pancreatic lineage are selected from the group consisting of definitive endoderm cells, primitive gut cells, posterior foregut cells, pancreatic endoderm cells and endocrine precursor cells.

In additional embodiments, the progenitor cells of the pancreatic lineage are selected from the group consisting of definitive endoderm cells, primitive gut cells, posterior foregut cells, pancreatic progenitor 1 (PP1) cells, endocrine pancreatic progenitor (PP2) cells and endocrine precursor cells (also called pancreatic endocrine progenitor cells).

In some embodiments, the kit further comprising one or more cell culture medium.

In some embodiments, the kit further comprises a plurality of differentiation factors for conducting a stepwise differentiation of pluripotent stem cells into the progenitor cells of the pancreatic lineage in a 2D cell culture prior to seeding on the scaffold. In some embodiments, the instruction manual further specifies instructions for carrying out a stepwise differentiation of pluripotent stem cells into the progenitor cells of the pancreatic lineage in a 2D cell culture prior to seeding on the scaffold.

In some embodiments, the kit further comprises agents and reagents for testing the beta cells following differentiation. For example, agents and reagents for testing insulin-secretion, agents and reagents for testing the expression of specific markers.

In some embodiments, when the kit comprises a scaffold that is not pre-seeded with cells, the scaffold may be lyophilized.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Differentiation of Pluripotent Stem Cells into Insulin-Producing Cells on a Lung Tissue-Derived Scaffold Versus Differentiation in a 2D Cell Culture—General Protocol

The process is carried out using porcine lung-derived acellular devitalized micro-organ matrices (MOMs) prepared as previously described (U.S. Pat. No. 10,093,896) and frozen until use.

Pluripotent stem cells, including induced human pluripotent stem cells (iPSCs) selected from iPSCs derived from adult cells, such as skin fibroblasts, lymphocytes and pancreatic cells, including beta cells, are suitable.

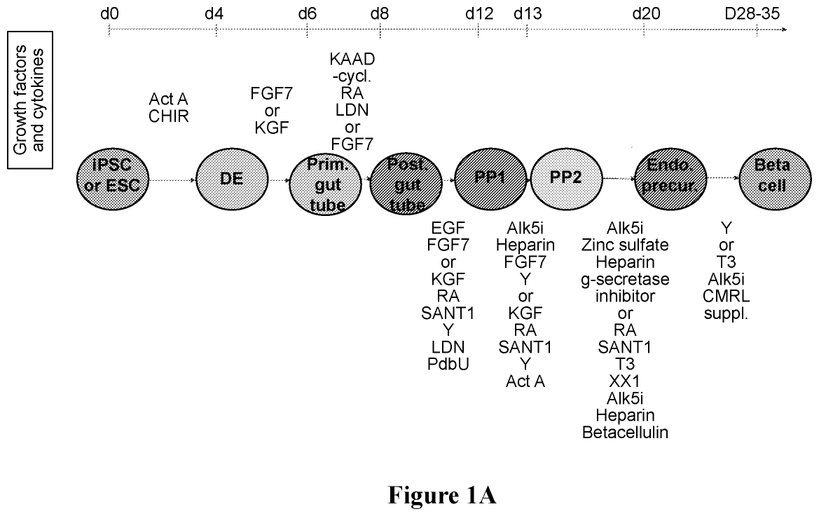

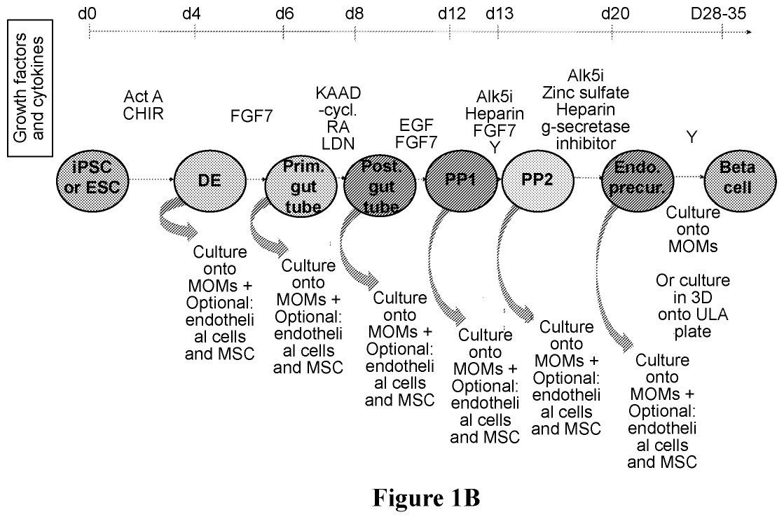

Standard differentiation protocols in a 2D culture are schematically illustrated in FIG. 1A. The procedure of the current example is illustrated in FIG. 1B.

In the current example the first step of the differentiation procedure, namely, differentiation from pluripotent stem cells into definitive endoderm (DE) cells, is carried out in a 2D culture, after which a sample of the DE cells is seeded on MOMs. Further steps are carried out in parallel, in the 2D culture and on MOMs. At each stage of differentiation, the cells are induced to the next stage of differentiation both in the 2D culture and on MOMs.

In addition, at each stage of the differentiation, a sample of cells from the 2D culture is taken and seeded on fresh MOMs, to continue the differentiation on the MOMs. The result is a series of MOM cultures, each containing cells that were seeded on the MOMs at a different stage of differentiation and completed the differentiation on the MOMs.

Step 1. Pluripotent Stage (Day 0)

2D Culture:

a. A plate is coated with Matrigel diluted 1/30 1 hour at 37° C.

b. Human pluripotent stem cells are seeded onto the Matrigel in mTeSR™ medium at 20-30% confluency

c. When the cells reach confluency of 80-90%, the cells are detached using TrypLE™ Express enzyme

d. The number of cells is counted

e. Cells are seeded in a 6-well plate coated with Matrigel™ diluted 1/30 1 hour at 37° C., 1,200,000 (1.2 million) cells per well in mTeSR™ medium

f. The cells are incubated for 24 h at 37° C., 5% CO₂.

Step 2. Definitive Endoderm (DE) Stage (Days 1-4)

2D Culture:

STEMdiff™ definitive endoderm kit is used (containing activin A+Chir99021, or GDF8+MCX-928, or activin A+Wnt3a).

a. Days 0-1: Definitive Endoderm Medium 1 is prepared according to the manufacturer's instructions (Supplements A and B are mixed) and added at 2 ml per well. The cells are incubated at 37° C., 5% CO₂.

c. Days 1-2.5: Definitive Endoderm Medium 2 is prepared according to the manufacturer's instructions (Supplement B is mixed with Definitive Endoderm Basal Medium (1:100)) and added at 2 ml per well (Medium 2 replaces Medium 1). The cells are incubated at 37° C., 5% CO₂.

d. Days 2.5-4: a fresh Definitive Endoderm Medium 2 is prepared (Supplement B is mixed with Definitive Endoderm Basal Medium (1:100)) and added at 2 ml per well. The cells are incubated at 37° C., 5% CO₂ for 36 h.

A sample of the obtained definitive endoderm (DE) cells is taken from the 2D culture and the cells are seeded on MOMs in a 12-well plate to obtain DE-MOMs, as follows:

10⁵ DE cells/MOM, at a density of 3 MOMs/well, in Definitive Endoderm Medium 2.

Cells from the 2D and MOM cultures are analyzed for the expression of one or more DE specific markers, preferably at least 2-3 markers, selected from: CXCR4, PDX1, FoxA2, c-kit, GP2, Sox17 and GSC.

Step 3. Primitive Gut (PG) Stage (Days 4-6)

The differentiation from DE to PG is carried out in parallel in the 2D culture and onto MOMs seeded with the DE cells (DE-MOMs), as follows:

a. The cells are washed with RPMI 1640 medium supplemented with GlutaMAX™ and containing 1% penicillin-streptomycin.

b. Primitive gut medium is added, containing:
i. RPMI 1640 medium+GlutaMAX™
ii. 1% PS (penicillin-streptomycin)
iii. 1% B27 supplement
iv. 50 ng/ml FGF7

c. The cells are incubated for 48 h at 37° C., 5% CO₂.

A sample of the obtained primitive gut (PG) cells is taken from the 2D culture and the cells are seeded on MOMs in a 12-well plate to obtain PG-MOMs, as follows:

10⁵ PG cells/MOM, at a density of 3 MOMs/well, in PG medium as above.

Cells from the 2D and MOM cultures are analyzed for the expression of one or more PG specific markers, preferably at least 2 markers, selected from: FoxA1, HNF1B, HNF4A.

Step 4. Posterior Foregut (PFG) Stage (Days 6-8)

The differentiation from PG to PFG is carried out in parallel in the 2D culture, on the DE-MOMs (which now contain PG cells) and on the PG-MOMs, as follows:

a. The PG medium is replaced with a posterior foregut medium added at 2 ml per well, containing
i. DMEM+GlutaMAX™
ii. 1% PS
iii. 1% B27
iv. 0.25 uM KAAD-cyclopamine
v. 2 uM retinoic acid
vi. 0.26 uM LDN-193189

b. The cells are incubated for 48 h at 37° C., 5% CO₂.

A sample of the obtained posterior foregut (PFG) cells is taken from the 2D culture and the cells are seeded on MOMs in a 12-well plate to obtain PFG-MOMs, as follows:

10⁵ PFG cells/MOM, at a density of 3 MOMs/well, in PFG medium as above.

Cells from the 2D and MOM cultures are analyzed for the expression of PFG specific markers: PDX1.

Step 5. Pancreatic Progenitor (PP1) Stage (Days 8-12)

The differentiation from PFG to PP1 is carried out in parallel in the 2D culture, on the DE-MOMs and PG-MOMs (which now contain PFG cells) and on the PFG-MOMs, as follows:

a. The PFG medium is replaced with a pancreatic progenitor 1 medium added at 2 ml per well, containing:
i. DMEM+GlutaMAX™
ii. 1% PS
iii. 1% B27
iv. 50 ng/ml EGF
v. 25 ng/ml FGF7

b. The cells are incubated for 48 h at 37° C., 5% CO₂

d. The medium is refreshed at Day 10 and the cells are incubated for additional 48 h at 37° C., 5% CO₂.

A sample of the obtained pancreatic progenitor (PP1) cells is taken from the 2D culture and the cells are seeded on MOMs in a 12-well plate to obtain PP1-MOMs, as follows:

10⁵ PP1 cells/MOM, at a density of 3 MOMs/well, in pancreatic progenitor 1 (PP1) medium as above.

Cells from 2D and MOM cultures are analyzed for the expression of one or more PP1 specific markers, preferably at least 2 markers, selected from: PDX1, HNF6, Prox1, Sox9.

Step 6. Endocrine pancreatic progenitor (PP2) stage (Days 12-13)

For the 2D culture, clusters for PP2 stage are prepared, as follows:

a. AggreWell™400 6-well plate is pretreated by adding 2 ml AggreWell™ Rinsing Solution.

b. The plate is centrifuged for 5 min at 1300 g

c. A PP2 cluster medium is prepared, containing:
i. RPMI+GlutaMAX™
ii. 1% PS
iii. 1% B27
iv. 1 uM ALK5 inhibitor Alk5 Receptor Inhibitor II (Alk5i II)
v. 10 ug/ml heparin
vi. 25 ng FGF7
vii. 10 uM Y-27632

d. The PP1 cells obtained in the previous step are detached from the plate with TrypLE™ Express

e. The cells are rinsed with the cluster medium and the cell suspension is transferred to a well of the AggreWell™400 6-well plate in 2 ml cluster medium.

g. The cells are incubated for 24 h at 37° C., 5% CO₂.

For the MOM cultures, the medium from the previous step (PP1 medium) is removed and replaced with the PP2 cluster medium. The MOMs are cultured for 24 hours at 37° C., 5% CO₂.

A sample of the obtained pancreatic progenitor (PP2) cells is taken from the AggreWell™ plate and the cells are seeded on MOMs in a 12-well plate to obtain PP2-MOMs, as follows:

10⁵ PP2 cells/MOM, at a density of 3 MOMs/well, in cluster medium as above.

Cells from the AggreWell™ plate and the MOM cultures are analyzed for the expression of one or more PP2 specific markers, preferably at least 2 markers, selected from: NKX6.1, PTF1A, NGN3, NKX2.2.

Step 7. Pancreatic Endocrine Progenitor (EN) Stage (Days 14-18)

For the 2D culture, the following is carried out:

a. A pancreatic endocrine progenitor stage medium is prepared, containing:
i. RPMI+GlutaMAX™
ii. 1% PS
iii. 1% B27
iv. 1 uM T3
v. 10 uM ALK5 inhibitor
vi. 10 uM zinc sulfate
vii. 10 ug/ml heparin
viii. 100 nm gamma secretase inhibitor, XXI
ix. 10 uM Y-27632

b. The clusters from the previous step are collected into a 50 ml tube, rinsed with 1 ml of the endocrine progenitor stage (EN) medium and pelleted by gravity

c. The pellet (clusters) is resuspend with the endocrine progenitor stage medium and the clusters are transferred to a low attachment 6-well plate

f. The plate is shaken and placed in a 37° C., 5% CO₂ incubator

g. The medium is refreshed every other day (clusters are collected and pelleted by gravity).

For the MOM cultures, the medium from the previous step (PP2 medium) is removed and replaced with the EN medium. The MOMs are cultured at 37° C., 5% CO₂. The medium is refreshed every other day.

A sample of the obtained pancreatic endocrine progenitor (EN) clusters is taken from the plate and the clusters are seeded on MOMs in a 12-well plate to obtain EN-MOMs, as follows:

50-100 EN clusters/MOM, at density of 3 MOMs/well, in EN medium as above.

Cells from the 2D and MOM cultures are analyzed for the expression of one or more EN specific markers, preferably at least 2-3 markers, selected from: PDX1, GP2, Nkx6.1, CHGA, INS, GCG and SST.

Step 8. Beta Cells Stage (Days 18-27)

The differentiation from EN to beta cells is carried out in parallel in the 2D culture and the MOMs cultures, as follows:

a. A pancreatic beta cell stage medium is prepared, containing:
i. RPMI+GlutaMAX™
ii. 1% PS
iii. 1% B27
iv. 10% FBS
v. 10 uM Y-27632

b. The cells are incubated at 37° C., 5% CO₂, the medium is refreshed every other day.

Cells from the 2D and MOM culture are analyzed for the expression of one or more beta cell specific markers, preferably at least 2-3 markers, more preferably at least 3-4 markers selected from: INS+, GCG-, PC1/3-, SST-, CHGA-, Pdx1+, GP2+, Nkx6.1+C-peptide and MAFA+.

### Example 2

### Differentiation of Pluripotent Stem Cells into Insulin-Producing Cells on a Lung Tissue-Derived Scaffold Versus Differentiation in a 2D Cell Culture-Specific Protocol

Porcine lung-derived acellular devitalized micro-organ matrices (MOMs) were prepared as previously described (U.S. Pat. No. 10,093,896) and frozen until use.

HES-2 cells were used.

Differentiation steps from pluripotent stem cells towards insulin-producing cells are illustrated schematically in FIG. 2.

In the current example, a complete differentiation process in a 2D culture was carried out as a control. In parallel, a differentiation process which begins in a 2D culture and is completed on MOMs was carried out. More particularly, differentiation up to Day 15 was carried out in a 2D culture, after which the differentiating cells were seeded on MOMs and the differentiation continued on the MOMs until Day 25.

Differentiation Protocol

M1 medium: MCDB131 (Gibco)+8 mM D-(+)-Glucose (Sigma)+1.23 g/L NaHCO₃(Sigma)+2% BSA (Sigma)+0.25 mM Vitamin C (SigmaAldrich)+1% Pen/Strep (Lonza)+1% L-glutamine (Lonza)

M2 medium: MCDB131+20 mM D-Glucose+1.754 g/L NaHCO₃+2% BSA+0.25 mM Vitamin C+Heparin 10 mg/ml (Sigma)+1% Pen/Strep+1% L-glutamine.

Prior to the differentiation process, the HES-2 cells were passaged once in an animal-component free cell culture medium, as follows:

a. A plate was coated with Matrigel™ diluted 1/30 1 hour at 37° C.

b. HES-2 cells were seeded onto the Matrigel in mTeSR™ medium at 20-30% confluency

c. When the cells reached confluency of 80-90%, the cells were detached using TrypLE™ Express enzyme

d. The number of cells was counted

e. Cells were seeded in a 6-well plate coated with Matrigel™ diluted 1/30 1 hour at 37° C., 1,200,000 (1.2 million) cells per well in mTeSR™ medium

f. The cells were incubated for 24 h at 37° C., 5% CO₂.

Days 0-3: Definitive Endoderm Stage

STEMdiff™ Definitive Endoderm Kit (STEMCELL), containing activin A, was used according to the manufacturer's instructions.

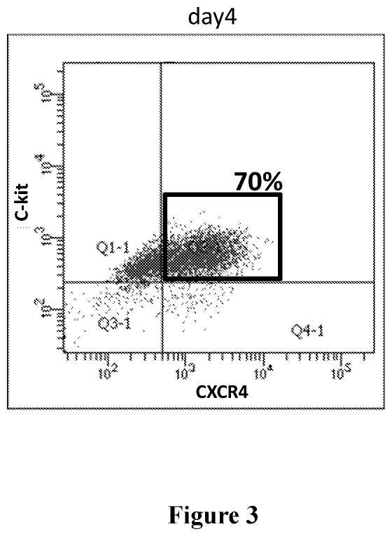

On Day 4, the cells were analyzed by FACS for expression of the definitive endoderm markers CXCR4 and c-kit. The results are shown in FIG. 3. As can be seen in the figure, definitive endoderm stage was reached at day 4 of differentiation, where approximately 70% of cells were positive for CXCR4 and c-Kit.

Days 4-6: Primitive Gut Stage

a. The cells were washed with M1 medium

b. Primitive gut medium was added (2 ml per well), containing:
i. M1 medium
ii. 50 ng/ml KGF (Peprotech)
iii. ITS-X supplement (Invitrogen) 1:50,000

c. The cells were incubated for 24 h at 37° C., 5% CO₂, after which the medium was changed to a fresh medium and the cells were incubated for additional 24 h at 37° C., 5% CO₂.

Day 4 cells were analyzed for the expression of FoxA2 and PDX1 by immunostaining and qPCR. The expression of FoxA2 and PDX1 was also tested on Day 9, to check for reduction in the expression of these two markers.

Days 7-8: Posterior Foregut Stage

a. The primitive gut medium was replaced with a posterior foregut medium added at 2 ml per well, containing:
i. M1 medium
ii. 50 ng/ml KGF
iii. 0.25 μM SANT-1 (Sigma)
iv. 21M retinoic acid (RA) (Sigma)
v. 200 nM LDN-193189 (only on Day 7) (Sigma)
vi. 500 nM PdBU (Millipore)
vii. ITS-X supplement 1:200

b. The cells were incubated for 24 h at 37° C., 5% CO₂, after which the medium was changed to a posterior foregut medium without LDN-193189 and the cells were incubated for additional 24 h at 37° C., 5% CO₂.

Days 9-13: Pancreatic Endoderm Stage

a. The posterior foregut medium was replaced with a pancreatic endoderm medium added at 2 ml per well, containing:
i. M1 medium
ii. 50 ng/ml KGF
iii. 0.25 μM SANT-1
iv. 100 nM RA
v. 2 μM iBET151 (Selleckchem)
vi. ITS-X supplement 1:200

b. The cells were incubated until Day 13 at 37° C., 5% CO₂, with daily medium exchanges.

Days 14-18: Endocrine Precursor Stage

a. The pancreatic endoderm medium was replaced with an endocrine precursor medium containing:
i. M2 medium
ii. 0.25 μM SANT-1
iii. 100 nM RA
iv. 1 μM PI 3-K Inhibitor XXI (Millipore)
v. 10 μM Alk5 Inhibitor II (Alk5i II) (Selleckchem)
vi. 1 μM L-3,30,5-Triiodothyronine (T3) (Sigma)
vii. 20 ng/ml Betacellulin (R&D)
viii. ITS-X supplement 1:200

b. The cells were incubated until Day 18 at 37° C., 5% CO₂ with daily medium exchanges. On Day 15, a sample of the cells differentiating in the 2D culture was taken and seeded on MOMs in a pancreatic endoderm medium as detailed above. The cells were seeded at a density of 50,000-100,000 cells per MOM, 3 MOMs per well. The next stages of the differentiation process were carried out in parallel in the 2D culture and on the MOMs.

Days 18-25: Beta Cell Stage

a. The endocrine precursor medium (in the 2D culture and in the MOM culture) was replaced with a beta cell medium containing:
i. CMRL medium
ii. 10% FBS
iii. 10 μM Alk5i II (Selleckchem)
iv. 1 μM L-3,30,5-Triiodothyronine (T3) (Sigma)
v. 10 mM nicotinamide (Sigma)

b. The cells were incubated until Day 25 at 37° C., 5% CO₂ with daily medium exchanges.

Clusters were generated between Day 21-Day 24. More particularly, the 2D culture was divided such that a portion of the cells continued to grow as a monolayer and another portion of the cells were grown as clusters. The clusters were generated as follows:
Removing medium
Adding 1 ml of TrypLE™ Express enzyme and waiting 2-3 min at room temperature
Removing TrypLE
Adding 2 ml of a beta cell medium per well (medium described in (a)) and pipetting up and down a few times to suspend the cells
Placing the cells in ultra-low attachment wells (each well that was collected from the 2D culture plate was transferred to a corresponding ultra-low attachment well).

In Vitro Assays

Insulin (INS) Expression

Expression of INS mRNA throughout the differentiation process was analyzed using real-time PCR. RNA extraction was performed using a Qiagen kit according to the kit's instructions. Reverse transcription was performed using a Quantabio kit according to the kit's instructions. qPCR was performed using SYBR® Green. In addition, insulin expression was also analyzed by immunofluorescence staining. To examine insulin-positive cells, cells were stained over night with anti-insulin antibody at 4° C. in PBS-Triton 0.5%. The following day cells were washed several times in PBS and stained with a secondary antibody (488) for 45 minutes at RT. Cells were washed several times with PBS and counterstained with DAPI.

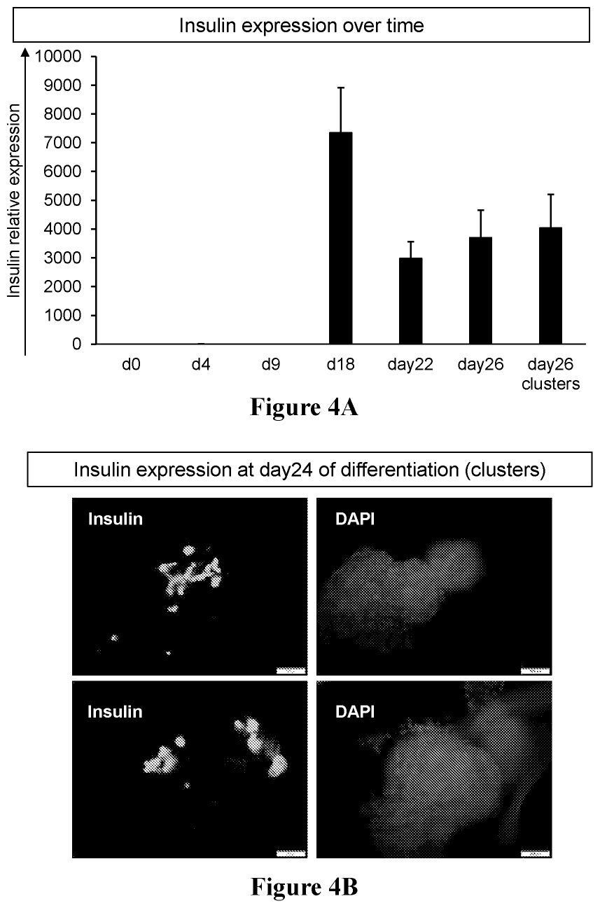

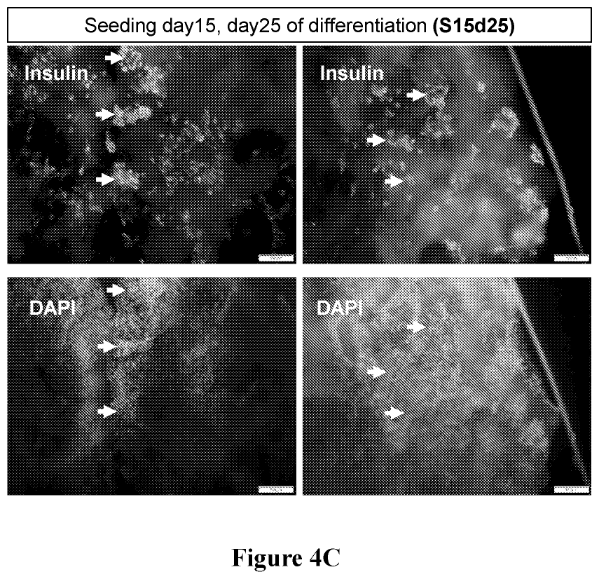

The results are summarized in FIGS. 4A-4C. FIG. 4A shows INS mRNA expression in cells that were differentiated in a 2D culture. The results pertain to cells grown as a monolayer unless “clusters” are indicated. As can be seen in the figure, insulin expression increased along the differentiation process, starting from Day 18. FIG. 4B shows insulin/DAPI staining of the clusters at Day 24. Some insulin-positive cells are found within the clusters at day 24 of differentiation. FIG. 4C shows insulin/DAPI staining of cells that completed the differentiation on MOMs. The figure shows staining at Day 25. MOMs improved beta cell differentiation and resulted in significantly more insulin-positive cells compared to the 2D culture, namely, significantly more cells express insulin when the differentiation was completed on MOMs as compared to differentiation carried out only in a 2D culture.

Glucose Stimulated Insulin Secretion (GSIS) Assay

Cells were treated with low glucose (LG, 2.5 mM glucose) for 20 minutes. Then, cells were treated with high glucose (HG, 11 mM glucose for additional 20 minutes and then with high glucose and KCL (both 11 mM) for additional 20 minutes (HG+KCL). Samples were collected at the starting point and every 10 minutes thereafter, until 60 minutes. Samples were then analyzed for insulin secretion via ELISA.

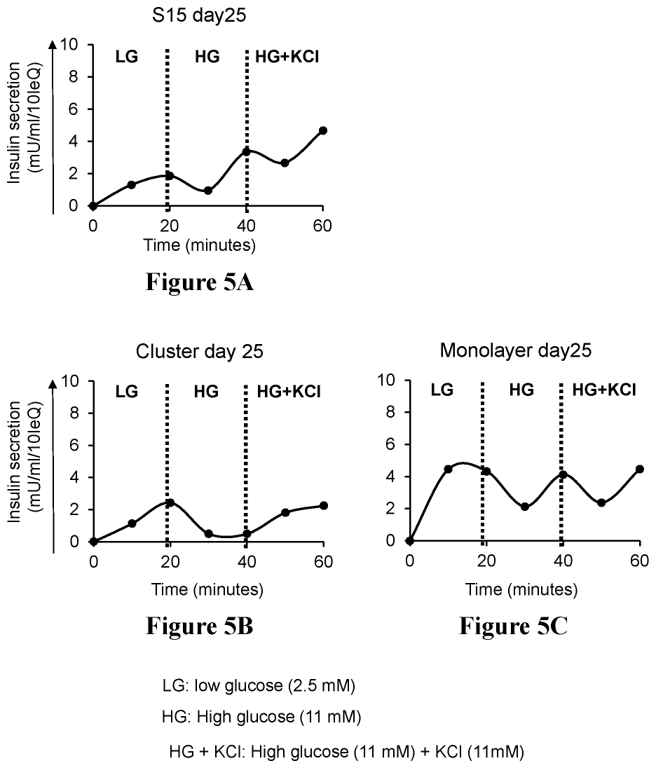

The results are shown in FIGS. 5A-5C. As can be seen in the figures, MOMs improved insulin secretion and regulation by the differentiated beta cells compared to the 2D culture. The differentiated beta cells that were seeded on MOMs on Day 15 and completed the differentiation on MOMs (FIG. 5A) secreted insulin in a regulated manner, while differentiated beta cells grown as clusters or monolayers in a 2D culture plate (FIGS. 5B-5C) did not show regulated insulin secretion.

As referred to herein, regulation of insulin secretion means that the amount of insulin secreted in LG is lower than the amount secreted in HG, and the amount secreted in HG is lower than the amount secreted in HG+KCL. It further describes a bi-phasic secretion of insulin in response to glucose. Increasing insulin secretion and displaying first- and second-phase insulin release to a high glucose challenge are key features of beta cell behavior.

### Example 3

### Differentiation of Pluripotent Stem Cells into Insulin-Producing Cells on a Lung Tissue-Derived Scaffold Versus Differentiation in a 2D Cell Culture-Modified Protocol

The protocol was modified for Days 0-3, which are carried out according to the protocol described below.

Day 0 Aggregation

Density of cells: 1 well should have approximately 1 million cells and there should be spaces between the pluripotent stem cell Matrigel colonies (50 to 60% confluence).

Media Needed:
Accutase™ (Gibco A11105 stock −20° C. store 4° C. cell culture)
   Stop medium (IMDM with L-glutamine and P/S: FCS; 50:50)
   IMDM with L-glutamine and P/S.
   D0 endoderm induction medium:

| | | Working | | |
|---|---|---|---|---|
| Reagent | Stock Conc. | conc | Use/ml | Volume |
| SFD | 100% | 100% | 1 ml/ml | 12 ml |
| Glutamine | 200 mM | 2 mM | 10 μl/ml | 120 μl |
| Ascorbic acid | 5 mg/ml | 50 μg/ml | 10 μl/ml | 120 μl |
| | | one use | | |
| Monothioglycerol | 1.25 gm/ml | 26 μl/2 ml | 3 ul/ml | 36 μl |
| (MTG) | | dilution | | |
| | | (4 × 10⁻⁴M) | | |
| | | one use | | |
| BMP4 | 10 μg/ml | 3 ng/ml | 0.3 ul/ml | 3.6 μl |

Differentiation Protocol

Day 0
Removing medium from wells
Adding 1 ml/well of Accutase™ (Gibco A11105 stock −20° C. store 4° C. cell culture) for 1 minute at room temperature
Aspirating Accutase™ off
Adding 1 ml/well of Stop medium+DNase (200 uL/12 ml)
Counting the cells
Scraping wells to produce clumps of cells, adding 1 ml/well of IMDM supplemented medium
Using a 10 ml pipette, pipetting 2-3 times
Adding solution from 6 wells into a 14 ml tube containing IMDM
Centrifuging 1200 rpm for 5 minutes
Resuspending in D0 endoderm induction medium and distributing 2 ml/well into a low cluster plate. Using 6-well Low Cluster plates, setting up the same number of plates as Matrigel plates or 6 wells/1 ten cm dish.
Incubating for 24 hours in a 5% O₂/5% CO₂/37° C. incubator.

Day 1: Endoderm Induction—Change Medium Completely
Removing embryoid bodies (EBs) from wells, putting into 14 ml tubes and allowing EBs to settle completely
Centrifuging 5 min at 1200 rpm
Preparing Day 1 endoderm induction medium and adding 1 ml of the Day 1 medium per well while EB's are settling:

| | Stock | Working | | |
|---|---|---|---|---|
| Reagent | Conc. | conc | Use/ml | Volume |
| SFD | 100% | 100% | 1 ml/ml | 12 ml |
| Glutamine | 200 mM | 2 mM | 10 μl/ml | 120 μl |
| Ascorbic acid | 5 mg/ml | 50 μg/ml one | 10 μl/ml | 120 μl |
| | | use | | |
| Monothioglycerol | 1.25 gm/ml | 26 μl/2 ml | 3 μl/ml | 36 μl |
| (MTG) | | dilution | | |
| | | (4 × 10⁻⁴M) | | |
| | | one use | | |
| BMP4 | 10 μg/ml | 0.5 ng/ml | 0.05 | 0.6 μl |
| | | | μl/ml | |
| bFGF | 10 μg/ml | 2.5 ng/ml | 0.25 | 3 μl |
| | | | μl/ml | |
| ActA | 10 μg/ml | 100 ng/ml | 10 μl/ml | 120 μl |

Incubating 48-72 hours in a 5% O₂/5% CO₂/37° C. incubator.

Day 4: Endoderm Induction—Change Medium Completely
Removing embryoid bodies (EBs) from wells, putting into 14 ml tubes and allowing EBs to settle completely
Preparing Day 4 endoderm induction medium and adding 1 ml of the Day 4 medium per well while EB's are settling (2 ml/well depending on density of cells):

| | Stock | Working | | |
|---|---|---|---|---|
| Reagent | Conc. | conc | Use/ml | Volume |
| SFD | 100% | 100% | 1 ml/ml | 12 ml |
| Glutamine | 200 mM | 2 mM | 10 μl/ml | 120 μl |
| Ascorbic acid | 5 mg/ml | 50 μg/ml | 10 μl/ml | 120 μl |
| | | one use | | |
| Monothioglycerol | 1.25 gm/ml | 26 μl/2 ml | 3 μl/ml | 36 μl |
| (MTG) | | dilution | | |
| | | (4 × 10⁻⁴M) | | |
| | | one use | | |
| VEGF | 5 μg/ml | 10 ng/ml | 2 μl/ml | 24 μl |
| ActA | 10 μg/ml | 100 ng/nl | 10 μl/ml | 120 μl |
| bFGF | 10 μg/ml | 2.5 ng/ml | 0.25 μl/ml | 3 μl |

Incubating 72 hours in a 5% O₂/5% CO₂/37° C. incubator.

### Example 4

### In Vitro Activity Assays

The beta cells obtained by differentiation onto MOMs are analyzed as follows and compared to beta cells differentiated in the 2D culture:
mRNA gene expression analysis of: (a) insulin (INS), glucagon (GCG) and pancreatic polypeptide (PPY); (b) maturity beta cell markers PDX1, NKX6.1, and MAFA; (c) cell glucose sensor genes (SLC2A1 and GCK); and (d) gap-junction genes (CDH1 and CX36). In addition, downregulation of pluripotency and progenitor genes is confirmed.
Immunochemistry analyses to confirm beta cell maturation: insulin (INS), glucagon (GCG) and pancreatic polypeptide (PPY), PDX1, NKX6.1, and MAFA.
In vitro pancreatic function evaluation by static and dynamic (perifusion assay) glucose stimulated insulin/C-peptide secretion assay (GSIS), followed by ELISA.
Quantification of insulin content after cell lysis and ELISA.
Ultrastructural analysis of differentiated beta cells by transmission/scanning electron microscopy (TEM/SEM) to examine the secretory vesicles contained within the cells.

### Example 5

### In Vivo Functional Assays

Beta cell-MOM compositions (5-10) are transplanted subcutaneously in immunocompromised mice to test their function in vivo. In particular, it is verified whether human insulin, and optionally also C-peptide, is detectable in the serum of animals transplanted with the beta cell-MOM compositions.

After a brief surgical recovery period (2 weeks), mice transplanted with the beta cell-MOM compositions are injected with glucose and serum is collected 30 min later. ELISA measurement of human insulin and optionally also C-peptide is performed to quantify human insulin/C-peptide secretion into the host's bloodstream.

To test whether beta cell-MOM compositions secrete insulin in response to glucose (GSIS in vivo), human insulin/C-peptide is measured in the bloodstream of a subset of mice both before (0 min) and after (30 min) an acute glucose challenge. Percentage of transplanted mice that show increased human insulin/C-peptide in the bloodstream after a glucose challenge, 2 weeks posttransplant, is calculated. As another measure of in vivo GSIS, the average ratio of insulin secreted after the glucose challenge compared to before is measured, and should preferably be >1. This in vivo stimulation index ranges from 0.4 to 4.3 for islet transplants.

Approximately 1 month post-transplantation animals are sacrificed and the engrafted beta cell-MOM compositions are removed for histology. IHC assay is carried out to check for C-peptide+/insulin+ cells.

Harvested grafts are also histologically evaluated for the following markers:
endocrine (Insulin, Glucagon and Somatostatin),
stemness (Sox2),
pancreatic endoderm (Pdx1, Nkx2.2 and Nkx6.1).

Beta cells with mature characteristics and morphological integration with surrounding tissues and functional integration between graft and site vascular network are evaluated (CD31 staining). The following histopathological assessments are carried out:

a. Degree of engraftment, viability of the cells

b. Degree of angiogenesis

c. Cellular infiltration into EMPs (H&E staining)

d. Fibrosis of the EMPs

e. Degradation of the EMPs

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for generating a population of insulin-producing beta cells, the method comprising:
(a) seeding progenitor cells of the pancreatic lineage on a devitalized, acellular, lung tissue-derived three-dimensional scaffold; and
(b) differentiating the progenitor cells of the pancreatic lineage to beta cells by a stepwise differentiation comprising sequentially applying a plurality of differentiation factors,
wherein the stepwise differentiation is carried out on the lung tissue-derived three-dimensional scaffold such that the cells remain on said scaffold throughout the differentiation process,
thereby generating a population of insulin-producing beta cells.

2. The method of claim 1, further comprising differentiating pluripotent stem cells to the progenitor cells of the pancreatic lineage in a 2D cell culture prior to step (a).

3. The method of claim 1, wherein the progenitor cells of the pancreatic lineage are selected from the group consisting of definitive endoderm cells, primitive gut cells, posterior foregut cells, pancreatic endoderm cells, pancreatic progenitor 1 (PP1) cells, endocrine pancreatic progenitor (PP2) cells and endocrine precursor cells.

4. The method of claim 1, wherein the progenitor cells of the pancreatic lineage are pancreatic endoderm cells, and wherein the sequentially applying a plurality of differentiation factors comprises:
(i) culturing the scaffold seeded with the pancreatic endoderm cells in a medium comprising one or more endocrine precursor differentiation factor, to obtain endocrine precursor cells on the scaffold; and
(ii) culturing the scaffold with the endocrine precursor cells in a medium comprising one or more beta cell differentiation factor, to obtain beta cells on the scaffold.

5. The method of claim 1, wherein the progenitor cells of the pancreatic lineage are pancreatic endoderm cells, and the method further comprises differentiating pluripotent stem cells to pancreatic endoderm cells in a 2D cell culture prior to step (a).

6. The method of claim 1, further comprising seeding at least one type of supporting cells selected from endothelial cells and mesenchymal stem cells (MSCs) on the scaffold concomitant with seeding the progenitor cells of the pancreatic lineage, and carrying out the differentiation process while the supporting cells are co-cultured with the differentiating cells on the scaffold.

7. (canceled)

8. A composition for generating insulin-producing beta cells, comprising:
(i) a devitalized, acellular, lung tissue-derived three-dimensional scaffold; and
(ii) progenitor cells of the pancreatic lineage seeded on said scaffold, wherein the progenitor cells of the pancreatic lineage are selected from the group consisting of definitive endoderm cells, primitive gut cells, posterior foregut cells, pancreatic endoderm cells, pancreatic progenitor 1 (PP1) cells, endocrine pancreatic progenitor (PP2) cells and endocrine precursor cells.

9. (canceled)

10. The composition of claim 8, wherein the progenitor cells of the pancreatic lineage are pancreatic endoderm cells.

11. The composition of claim 8, further comprising at least one type of supporting cells selected from endothelial cells and mesenchymal stem cells (MSCs) seeded on the scaffold.

12. (canceled)

13. A method for generating insulin-producing beta cells, comprising:
(a) providing the devitalized, acellular, lung tissue-derived three-dimensional scaffold seeded with the progenitor cells of the pancreatic lineage according to claim 8; and
(b) differentiating the progenitor cells of the pancreatic lineage to beta cells by a stepwise differentiation, wherein the stepwise differentiation is carried out on the lung tissue-derived three-dimensional scaffold such that the differentiating cells remain on said scaffold throughout the differentiation process.

14. The method of claim 13, wherein the scaffold in step (a) is further seeded with at least one type of supporting cells selected from endothelial cells and mesenchymal stem cells (MSCs), and wherein the stepwise differentiation is carried out on the scaffold in the presence of the supporting cells.

15-23. (canceled)

24. A method for producing an artificial micro-organ, the method comprising:
(a) seeding progenitor cells of the pancreatic lineage on a devitalized, acellular, lung tissue-derived three-dimensional scaffold; and
(b) differentiating the progenitor cells of the pancreatic lineage to insulin-producing beta cells by a stepwise differentiation in which a plurality of differentiation factors are sequentially applied, wherein the stepwise differentiation is carried out on the lung tissue-derived three-dimensional scaffold such that the cells remain on said scaffold throughout the differentiation process,
thereby obtaining an artificial micro-organ comprising the insulin-producing beta cells cultured on the lung tissue-derived three-dimensional scaffold and maintaining glucose-responsive insulin secretion when cultured on said scaffold.

25. The method of claim 24, wherein the progenitor cells of the pancreatic lineage are selected from the group consisting of definitive endoderm cells, primitive gut cells, posterior foregut cells, pancreatic endoderm cells, pancreatic progenitor 1 (PP1) cells, endocrine pancreatic progenitor (PP2) cells and endocrine precursor cells.

26. The method of claim 24, further comprising seeding at least one type of supporting cells selected from endothelial cells and mesenchymal stem cells (MSCs) on the scaffold, and carrying out the differentiation process while the supporting cells are co-cultured with the differentiating cells on the scaffold.

27. (canceled)

28. An artificial micro-organ comprising a lung tissue-derived three-dimensional scaffold and insulin-producing beta cells cultured thereon, produced by the method of claim 24.

29. A method for treating diabetes in a subject in a need thereof, the method comprising transplanting in the subject a therapeutically effective amount of an artificial micro-organ produced by the method of claim 24.

30. The method of claim 29, wherein the diabetes is type I diabetes.

31. The method of claim 29, wherein the diabetes is type II diabetes.

32. The method of claim 29 wherein the diabetes is caused by pancreas inflammation or other causes to pancreas disfunction.

33. The method of claim 29, wherein the source of progenitor cells of the pancreatic lineage is autologous to the treated subject.
